SEQUENCE LISTING (44 sequences)

SEQ ID NO: 1 - protein, 11 aa, synthetic construct
  REGION 1..11: note = Albumin binding peptide (PEP)
DICLPRWGCLW

SEQ ID NO: 2 - protein, 5 aa, synthetic construct
  REGION 1..5: note = linker peptide
GGGGS

SEQ ID NO: 3 - protein, 6 aa, synthetic construct
  REGION 1..6: note = linker peptide
GGSGSS

SEQ ID NO: 4 - protein, 5 aa, synthetic construct
  REGION 1..5: note = linker peptide
EAAAK

SEQ ID NO: 5 - protein, 46 aa, synthetic construct
  REGION 1..46: note = linker peptide
AEAAAKEAAAKEAAAKEAAAKALEAEAAAKEAAAKEAAAKEAAAKA

SEQ ID NO: 6 - protein, 18 aa, synthetic construct
  REGION 1..18: note = linker peptide
KESGSVSSEQLAQFRSLD

SEQ ID NO: 7 - protein, 14 aa, synthetic construct
  REGION 1..14: note = linker peptide
EGKSSGSGSESKST

SEQ ID NO: 8 - protein, 12 aa, synthetic construct
  REGION 1..12: note = linker peptide
GSAGSAAGSGEF

SEQ ID NO: 9 - protein, 20 aa, synthetic construct
  REGION 1..20: note = E2A sequence
QCTNYALLKLAGDVESNPGP

SEQ ID NO: 10 - protein, 22 aa, synthetic construct
  REGION 1..22: note = F2A sequence
VKQTLNFDLLKLAGDVESNPGP

SEQ ID NO: 11 - protein, 18 aa, synthetic construct
  REGION 1..18: note = T2A sequence
EGRGSLLTCGDVEENPGP

SEQ ID NO: 12 - protein, 19 aa, synthetic construct
  REGION 1..19: note = P2A sequence
ATNFSLLKQAGDVEENPGP

SEQ ID NO: 13 - protein, 4 aa, synthetic construct
  REGION 1..4: note = caspase cleavage site
DEVD

SEQ ID NO: 14 - protein, 4 aa, synthetic construct
  REGION 1..4: note = caspase cleavage site
DLDV

SEQ ID NO: 15 - protein, 4 aa, synthetic construct
  REGION 1..4: note = caspase cleavage site
DEID

SEQ ID NO: 16 - protein, 4 aa, synthetic construct
  REGION 1..4: note = caspase cleavage site
DEHD

SEQ ID NO: 17 - protein, 4 aa, synthetic construct
  REGION 1..4: note = caspase cleavage site
DKAD

SEQ ID NO: 18 - protein, 4 aa, synthetic construct
  REGION 1..4: note = caspase cleavage site
DSFD

SEQ ID NO: 19 - protein, 4 aa, synthetic construct
  REGION 1..4: note = caspase cleavage site
DSSD

SEQ ID NO: 20 - protein, 4 aa, synthetic construct
  REGION 1..4: note = caspase cleavage site
DGKD

SEQ ID NO: 21 - protein, 4 aa, synthetic construct
  REGION 1..4: note = caspase cleavage site
DYND

SEQ ID NO: 22 - protein, 4 aa, synthetic construct
  REGION 1..4: note = caspase cleavage site
DRPD

SEQ ID NO: 23 - protein, 4 aa, synthetic construct
  REGION 1..4: note = caspase cleavage site
DNVD

SEQ ID NO: 24 - protein, 4 aa, synthetic construct
  REGION 1..4: note = caspase cleavage site
VQVD

SEQ ID NO: 25 - protein, 4 aa, synthetic construct
  REGION 1..4: note = caspase cleavage site
LETD

SEQ ID NO: 26 - protein, 4 aa, synthetic construct
  REGION 1..4: note = caspase cleavage site
LEHD

SEQ ID NO: 27 - protein, 4 aa, synthetic construct
  REGION 1..4: note = caspase cleavage site
WEHD

SEQ ID NO: 28 - protein, 6 aa, synthetic construct
  REGION 1..6: note = caspase cleavage site
ELQTDG

SEQ ID NO: 29 - protein, 6 aa, synthetic construct
  REGION 1..6: note = caspase cleavage site
RIEADS

SEQ ID NO: 30 - protein, 5 aa, synthetic construct
  REGION 1..5: note = caspase cleavage site
VDVAD

SEQ ID NO: 31 - protein, 4 aa, synthetic construct
  REGION 1..4: note = caspase cleavage site
DFRD

SEQ ID NO: 32 - protein, 6 aa, synthetic construct
  REGION 1..6: note = caspase cleavage site
KGDEVD

SEQ ID NO: 33 - protein, 6 aa, synthetic construct
  REGION 1..6: note = caspase cleavage site
RGDEVD

SEQ ID NO: 34 - protein, 11 aa, synthetic construct
  REGION 1..11: note = caspase cleavage site
CRGDCGGDEVD

SEQ ID NO: 35 - protein, 5 aa, synthetic construct
  REGION 1..5: note = caspase cleavage site
DEVDR

SEQ ID NO: 36 - protein, 10 aa, synthetic construct
  REGION 1..10: note = caspase cleavage site
CQRPPRDEVD

SEQ ID NO: 37 - protein, 4 aa, synthetic construct
  REGION 1..4: note = cathepsin cleavage site
GRRG

SEQ ID NO: 38 - protein, 4 aa, synthetic construct
  REGION 1..4: note = cathepsin cleavage site
FRRG

SEQ ID NO: 39 - protein, 4 aa, synthetic construct
  REGION 1..4: note = cathepsin cleavage site
ARRG

SEQ ID NO: 40 - protein, 5 aa, synthetic construct
  REGION 1..5: note = cathepsin cleavage site
KGRRG

SEQ ID NO: 41 - protein, 6 aa, synthetic construct
  REGION 1..6: note = cathepsin cleavage site
RGDRRG

SEQ ID NO: 42-44 - skipped (3 entries)

(19) United States
(12) Patent Application Publication
(10) Pub. No.: US 2025/0195661 A1
(43) Pub. Date: Jun. 19, 2025

(54) NIR-responsive stem cell-derived inflammation attenuating complex and use thereof

(71) Applicants: ECTOSOME INC., Incheon (KR); Gachon University of Industry-Academic cooperation Foundation, Seongnam-si (KR)

(72) Inventors: Dongwoo KHANG, Seongnam-si (KR); Min Jun SHIN, Seongnam-si (KR); Junyoung PARK, Seongnam-si (KR); Su Hyun LIM, Seongnam-si (KR)

(21) Appl. No.: 18/979,865

(22) Filed: Dec. 13, 2024

(30) Foreign Application Priority Data
Dec. 15, 2023 (KR) 10-2023-0183725

(51) Int. Cl. A61K 41/00; A61K 31/573; A61K 33/242; A61K 35/28; A61K 47/69; A61P 19/02

(52) U.S. Cl. CPC A61K 41/0052; A61K 31/573; A61K 33/242; A61K 35/28; A61K 47/6901; A61K 47/6929; A61P 19/02

(57) ABSTRACT

Provided is an inflammation attenuating complex, which uses infrared-responsive photothermal stem cells that effectively treat inflammatory diseases by efficiently delivering anti-inflammatory drugs to the site of inflammation due to enhanced migration ability to the site of inflammation in the body, and a use thereof.

### STATEMENT OF GOVERNMENT-SPONSORED RESEARCH

This invention was supported by Bio Medical Technologies Development Project under Grant Nos. 1711162167 and 711168722 awarded by Ministry of Science and ICT of Republic of Korea.

### CROSS-REFERENCE TO RELATED APPLICATION

This application claims priority to Korean Patent Application No. 10-2023-0183725 filed on Dec. 15th 2023 and all the benefits accruing therefrom under 35 U.S.C. § 119, the contents of which are incorporated by reference in their entirety.

### REFERENCE TO AN ELECTRONIC SEQUENCE LISTING

The contents of the electronic sequence listing (SPJ20245295US_SEQ.xml; Size: 52,516 bytes; and Date of Creation: Dec. 10, 2024) is herein incorporated by reference in its entirety. The contents of the electronic sequence listing in no way introduces new matter into the specification.

### TECHNICAL FIELD

The present disclosure relates to an inflammation attenuating complex using a near-infrared (NIR)-responsive photothermal stem cells, and more particularly, to an inflammation attenuating complex using NIR-responsive photothermal stem cells, and a use thereof.

### BACKGROUND

Rheumatoid arthritis (RA) is a systemic autoimmune disease in which loss of immunological self-tolerance causes chronic inflammation in the joints, followed by destruction of cartilage and bone, thereby resulting in worsening pain and stiffness (J. S. Smolen et al., Nat. Rev. Dis. Primers. 4:18001, 2018). Despite significant improvements in the management of patients with RA over the past several decades, a significant proportion of patients with RA remain refractory to treatment and present progression of residual disease after failure of disease-modifying antirheumatic drugs, leaving no alternatives (A. Latourte et al., Nat. Rev. Rheumatol. 16 (12): 673-688, 2020). Osteoarthritis is the most common degenerative joint disease in which cartilage and joints deteriorate over time, often resulting in chronic pain and stiffness, and about 300 million people are suffering from the disease worldwide. Disease-modifying drugs or treatments that reduce symptoms other than slowing or stopping the progression of the disease have not been approved by regulatory agencies; for example, only symptom-relieving drugs, such as pain relievers for OA, can be used at present (J. Martel-Pelletier et al., Nat, Rev, Dis, Primers, (2), 16072). Therefore, the biomedical community has focused scientific efforts and resources on resolving arthritis from the aspects of tissue remodeling and repair (Y. Han et al., Signal Transduct. Target. Ther. 7 (1): 92, 2022). However, tissue remodeling is not mediated by a single effector, but rather by the complex regulation of various factors that maintain homeostasis. In this regard, stem cell research has received considerable attention due to the regulatory ability of stem cells to regenerate damaged tissues in joint diseases including OA and RA. Among the above-described stem cells, mesenchymal stem cells (MSCs) are considered a promising therapeutic vector for the treatment of arthritis due to the ease of their isolation and preparation and innate anti-inflammatory response ability. Additionally, the clinical use of MSCs is relatively free from ethical concerns and teratoma formation (tumors resulting from abnormal development of pluripotent stem cells) (H. H. Wu et al., Int. J. Nanomedicine 2021:16 8485-9446, 2021).

### SUMMARY

However, in the case of relevant art, there is a limitation of causing safety issues due to genetic modification and poor ability to migrate to inflamed cells.

The present disclosure aims to resolve a number of issues, including the above-mentioned issues, and the present disclosure provides an inflammation attenuating complex using NIR-responsive photothermal stem cells that effectively treat inflammatory diseases including arthritis by efficiently delivering anti-inflammatory drugs to the site of inflammation due to their enhanced ability to migrate to the site of inflammation in the body, and a use thereof. However, these provisions are exemplary and do not limit the scope of the present disclosure.

In accordance with an exemplary embodiment, there is provided a stem cell-gold nanoparticle (AuNP) complex, in which gold nanoparticles (AuNPs) loaded with an anti-inflammatory agent are bound to a surface of stem cells.

In accordance with another exemplary embodiment, there is provided a drug delivery carrier comprising the stem cell-gold nanoparticle complex as an effective ingredient.

In accordance with still another exemplary embodiment, there is provided a pharmaceutical composition for treating arthritis comprising the stem cell-gold nanoparticle complex as an effective ingredient.

In accordance with still another exemplary embodiment, there is provided a composition for treating pain caused by joint inflammation comprising the stem cell-gold nanoparticle complex as an effective ingredient.

In accordance with still another exemplary embodiment, there is provided a method of treating arthritis in a subject in need of comprising administrating therapeutically effective amount of the stem cell-gold nanoparticle complex to the subject.

In accordance with still another exemplary embodiment, there is provided a method of relieving pain caused by joint inflammation in a subject comprising administrating therapeutically effective amount of the stem cell-gold nanoparticle complex to the subject.

### BRIEF DESCRIPTION OF THE DRAWINGS

Exemplary embodiments can be understood in more detail from the following description taken in conjunction with the accompanying drawings, in which:

FIG. 1a is a schematic diagram showing an inflammation-mediated education procedure for MSCs and changes resulting from the procedure. The inflammation-mediated education enhances the ability of MSCs to target inflamed FLSs and macrophages;

FIG. 1b is a graph showing the analysis results of the differential in vitro tropism for J774 (macrophages) and FLSs based on the MSC lineage. J774 and FLSs were stimulated with 50 ng/mL LPS and 20 ng/mL TNF-α, respectively. ADMSCs exhibit accelerated homing ability toward stimulated J774 and FLSs. The data represent mean±SEM (n=5) and p-value criterion is the control group (MSC migration group toward CTL and MSCs);

FIG. 1c is a graph showing the analysis results of the gene expression of chemokine receptors directly related to MSC migration. The gene expression was upregulated after education. The data represent mean±SEM (n=5);

FIG. 1d is a graph showing the analysis results of the improved migration ability of ADMSCs after education. The educated ADMSCs exhibit enhanced migration toward stimulated FLSs and J774 compared to naïve ADMSCs. The data represent mean±SEM (n=5) and p-values refer to the migration of naïve ADMSCs to ADMSCs;

FIG. 1e shows microscopic images (top) and graphs (bottom) illustrating the degree of migration after treating the MSCs of the present disclosure with antibodies. After blocking chemokine receptors on the surface of MSCs, naïve and educated ADMSCs migrated toward ADMSCs and toward stimulated FLSs or J774. MSCs were treated along with anti-IgG, anti-CCR1, anti-CCR2, anti-CCR3, and anti-CXCR4 antibodies. The migration of MSCs to FLSs and J774 was reduced after blocking CCR2, CCR3, or CXCR4. The data represent mean±SEM (n=5) and p-values are relative to naïve ADMSCs. *p<0.0332, **p<0.021, ***p<0.0002, and ****p<0.0001;

FIG. 2a is a schematic diagram illustrating the analysis procedure for the inflamed joint targeting ability of MSCs through a education procedure, analyzing the ability of educated MSCs to target inflamed joints;

FIG. 2b shows images (top) and graphs (bottom) illustrating whole mouse IVIS imaging results by observing the improvement in the in vivo migration ability of MSCs after education;

FIG. 2c shows micrographic images (top) and graphs (bottom) illustrating the enhancement of the in vivo migration ability of MSCs after education in the mice possessed. The mice were sacrificed 3 days after the first intravenous injection of 680-labeled MSCs into the mice with established collagen-induced arthritis, and joint tissues were collected for histological analysis. The images on top are confocal s of MSCs migrating through an inflamed joint. The data represent mean±SEM (n=3). *p<0.0332 and ***p<0.0002;

FIG. 3a shows a Venn diagram illustrating changes in the expression of inflammation-related and migration-related genes in educated MSCs. The Venn diagram shows RNA sequencing for gene expression profiling of MSCs after training with J774 conditioned medium (CM), FLS CM, and a mixed CM (J774+FLS). The Venn diagram represents the intersection of differentially upregulated and downregulated genes;

FIG. 3b shows a graph illustrating the analysis results of gene ontology (GO) term enrichment (biological functions) of MSCs after the mixed CM training. In the gene category group, 15 pre-defined GO terms differentially expressed genes into functional groups;

FIG. 3c shows graphs illustrating the heatmap results for the expression levels of inflammation-related genes among a total of 2,123 transcripts that were upregulated or downregulated in all three groups (J774 CM, FLS CM, and mixed CM);

FIG. 3d shows a graph illustrating the heatmap analysis results of the expression levels of chemokine receptors (migration-related genes) among a total of 2,123 transcripts that were upregulated or downregulated in all three groups (J774 CM, FLS CM, and mixed CM);

FIG. 4a shows a schematic diagram illustrating the analysis of binding between gold nanostar (AuS)-triamcinolone (TA) and Edu-MSC, showing an embodiment of the chemical binding of gold nanostar (AuS)-triamcinolone (TA). The PEGylated AuS (AuS-PEG) non-covalently bound to the TA is shown;

FIG. 4b shows a schematic diagram illustrating binding of Edu-MSC and AuS-CD90-TA using CD90 and anti-CD90 antibodies (Abs);

FIG. 4c shows micrographic images illustrating the measurement results obtained using energy-dispersive X-ray spectroscopy mapping, showing the distributions of Au (yellow), S (elements of PEG and cysteamine, red), and F (element of TA, blue) determined using energy-dispersive X-ray. Scale bar=20 nm;

FIG. 4d shows images (top) and a graph (bottom) illustrating thermal images of photoactivation of AuS-TA by near-infrared (NIR) laser irradiation. The temperature difference (ΔT) was calculated using the difference in temperature between the distilled water and AuS-TA points;

FIG. 4e shows representative confocal images illustrating binding of Edu-MSC and AuS-CD90-TA of the present disclosure. The representative confocal images show adjacent locations of AuS-TA (green), anti-CD90 Ab (yellow), and Edu-MSC (red). The inserted image shows a magnified view of the box. The scale bar is 20 μm. A higher magnification image of AuS-TA bound to Edu-MSCs by CD90 molecules is shown on the right. White arrows highlight the evidence for surface localization of AuS-TA using CD90 molecules. The 3-dimensional analysis of confocal images shows the localization of AuS-TA (green) on the surface of Edu-MSCs;

FIG. 4f shows a graph illustrating the results of ultraviolet-visible spectroscopy analysis of AuS-TA. As confirmed in AuS-TA, it shows a TA peak at 242 nm and an AuS peak at 810 nm;

FIG. 4g shows a graph illustrating the results of UV-Vis spectroscopy analysis of Edu-MSCs-AuS-TA. The UV-visible spectroscopy results showing TA peak at 242 nm, Edu-MSCs peak at 260 nm, AuS peak at 810 nm, and peaks of Edu-MSCs-AuS-TA at 230 nm (shifted from the Edu-MSCs peak at 242 nm) and 250 nm (shifted from the AuS-TA peak at 260 nm) confirmed by the binding of Edu-MSCs and AuS-TA;

FIG. 5a shows schematic diagrams illustrating the endocytosis analysis results of AuS-TA and Edu-MSCs-AuS-TA, which show the endocytosis pathway analysis and intracellular trafficking of AuS-TA in FLS derived from OA patients and RA patients;

FIG. 5b shows graphs illustrating the analysis results of the endocytosis pathway of AuS-TA in FLS. FLS were stimulated with TNF-α at 20 ng/mL, and the endocytosis pathway was inhibited by CPZ (clathrin-mediated endocytosis) at 20 μM, GEN (caveolin-mediated endocytosis) at 200 μM, and EIPA (a macrophage action) at 25 μM. The fluorescence intensity of AuS-TA (Alexa488+) analyzed in FLS is shown. The data represent mean±SEM (n=3);

FIG. 5c shows fluorescence micrographic images illustrating the analysis results of intracellular exchange of AuS-TA in FLS. AuS-TA was labeled with Alexa 488. Early endosomes (EE) and late endosomes (LE) were stained with EEA-1 antibody (Alexa 555, red) and M6PR antibody (Alexa 594, purple), respectively;

FIG. 5d shows schematic diagrams illustrating the intracellular exchange and endocytosis pathway analysis of Edu-MSCs-AuS-TA in FLS (left) and a confocal image of Edu-MSCs-AuS-TA delivering AuS-TA to FLS (right). AuS-TA and FLS were labeled with Alexa 488 and DiD, respectively;

FIG. 5e shows graphs illustrating the analysis results of endocytosis pathway of Edu-MSCs-AuS-TA in FLS. The data represent mean±SEM (n=3);

FIG. 5f shows fluorescence micrographic images illustrating the analysis results of intracellular exchange of Edu-MSCs-AuS-TA in FLS. ns<0.1234, *p<0.0332, **p<0.021, ***p<0.0002, and ****p<0.0001;

FIG. 6a shows a schematic diagram illustrating the inactivation of RA patient-derived FLS and OA patients using Edu-MSCs-AuS-TA, in which the immunomodulatory efficacy of Edu-MSCs-AuS-TA in RA FLS and OA FLS is shown;

FIG. 6b shows graphs illustrating the analysis results of the expression of cytokines in RA patient-derived FLS by the treatment of Edu-MSC-AuS-TA of the present disclosure. These graphs show downregulation of the expression of pro-inflammatory cytokines (TNF-α, IL1B, and IL6) and upregulation of the expression of anti-inflammatory cytokines (IL4, IL10, and Arg-1) in RA patient-derived FLS by Edu-MSC, AuS-TA, and Edu MSC-AuS-TA. The data represent mean±SEM (n=3), and p-value criterion is unstimulated RA patient-derived FLS (NC);

FIG. 6c shows graphs illustrating the analysis results of the expression of cytokines in OA patient-derived FLS by the treatment of Edu-MSC-AuS-TA of the present disclosure. These graphs show downregulation of the expression of pro-inflammatory cytokines (TNF-α, IL1B, and IL6) and upregulation of the expression of anti-inflammatory cytokines (IL4, IL10, and Arg-1) in OA patient-derived FLS by Edu-MSC, AuS-TA, and Edu MSC-AuS-TA. The data represent mean±SEM (n=3), and p-value criterion is unstimulated OA patient-derived FLS (NC). ns<0.1234, *p<0.0332, **p<0.021, ***p<0.0002, and ****p<0.0001;

FIG. 7a shows a schematic diagram illustrating the plan for establishing a CIA model and the Edu-MSCs-AuS-TA treatment, in which the anti-inflammatory efficacy and cartilage protective ability of Edu-MSCs-AuS-TA of the present disclosure for moderate arthritis is analyzed. Arthritis was induced in 6-week-old DBA/1 male mice by two injections of collagen. 28 Days after the first injection of collagen, the CIA model with moderate arthritis (arthritis severity score <4) showed a significant reduction in the incidence of osteoarthritis using Edu-MSCs-AuS-TA via the intraperitoneal route;

FIG. 7b shows the images illustrating the observation results after the treatment of Edu-MSC-AuS-TA of the present disclosure on the feet of experimental mice. The images are representative images by hematoxylin-eosin staining and safranin-O staining of the feet of ankle joints of the mice, treated with saline, Edu-MSCs, AuS-TA, Edu-MSC+AuS-TA (a mixture), and Edu MSC-AuS-TA (a conjugate);

FIG. 7c shows a graph illustrating the analysis results of the paw edema score during CIA establishment in DBA/1 mice;

FIG. 7d shows histological images of the cartilage observed under a microscope (top) and a graph illustrating the analysis results of the modified OARSI score (bottom). The data represent the mean±standard error of the mean;

FIG. 8a shows a schematic diagram illustrating the plan for establishing a CIA model and the procedure for administering Edu-MSCs-AuS-TA via photothermal therapy (PTT), in which the therapeutic efficacy of Edu-MSCs-AuS-TA for advanced arthritis was examined using PTT. Arthritis was induced in 6-week-old DBA/1 male mice by two injections of collagen. 42 Days after the first injection of collagen, the CIA model with advanced arthritis stage (arthritis severity score>8) showed a significant reduction using Edu-MSCs-AuS-TA via the intraperitoneal route and after NIR laser irradiation on the inflamed joint;

FIG. 8b shows a graph illustrating the analysis results of the arthritis severity score according to the administration of Edu-MSCs-AuS-TA of the present disclosure and NIR laser irradiation. Paw edema scores were analyzed during the CIA establishment in DBA/1 mice in the presence of saline+laser, Edu-MSCs+AuS TA (a mixture), a mixture+laser, Edu-MSCs-AuS-TA (a conjugate), and a conjugate+laser. The data represent the mean±standard error of the mean. (n=5). ns<0.1234, *p<0.0332, **p<0.021, and ****p<0.0001;

FIG. 8c shows images observed after laser irradiation on the foot of a mouse animal model. The foot images and thermal images of the laser-irradiated joint in the CIA model treated with conjugate+laser before treatment (left, score=4) and after treatment (right, score=1) are shown;

FIG. 8d shows Micro-CT images of the hindpaw in the CIA mouse model;

FIG. 9a shows a schematic diagram illustrating the downregulated IL22R expression in T cells by PTT using AuS-TA and NIR laser irradiation, in which the mechanism of T cell regulation by PTT along with downregulation of IL22R expression;

FIG. 9b shows graphs illustrating the analysis results of the expression level of IL22R mRNA due to the effect of PTT (AuS-TA+laser). Expression levels of IL22R and CD23 transcripts were normalized to the expression level of GAPDH transcript. The data represent the mean±SEM of three independent experiments (n=3);

FIG. 9c shows gel images illustrating the results of Western blot analysis of cell lysates examined for IL22R, beta-actin, and membrane housekeeping protein (Na-K ATPase);

FIG. 9d shows a schematic diagram illustrating the inhibitory effect of Th17 differentiation by PTT;

FIG. 9e shows graphs illustrating the results of FACS analysis of the Th17 population. The downregulation of IL22R expression in T cells by PTT showed an inhibitory effect on Th17 differentiation;

FIG. 9f shows a graph illustrating the analysis results of the expression level of Th17;

FIG. 10a shows a schematic diagram illustrating the anti-inflammatory efficacy of Edu-MSCs-AuS-TA through repolarization of macrophages to an anti-inflammatory phenotype (M2) and inhibition of neutrophil recruitment, in which the effects of inducing macrophage repolarization images and inhibiting neutrophil recruitment in the synovium were confirmed;

FIG. 10b shows confocal images of CD86 (M1 marker) and Dectin-1 (M2 marker) in ankle joints treated with various drugs (top) and the analysis results of intensity thereof (bottom). The group treated with Edu-MSCs-AuS-TA showed macrophage repolarization from pro-inflammatory M1 (a decreased level of CD86 which is an M1 marker) to an anti-inflammatory M2 (an increased level of Dectin-1 which is an M2 marker) phenotype. The graph of the average fluorescence intensity ratio of M1 and M2 signals within the box visualized macrophage repolarization from M1 to M2 in the group treated with Edu-MSCs-AuS-TA;

FIG. 10c shows graphs showing the analysis results of fluorescence-activated cell sorting of neutrophils in synovial tissues of mice subjected to various treatments, confirmed using Gr-1 and CD11b. Edu-MSCs-AuS-TA effectively inhibited the influx of neutrophils into the joints;

FIG. 11a shows a schematic diagram illustrating a series of behavioral test procedures using a mouse animal model for the evaluation of RA-related pain;

FIG. 11b shows graphs illustrating the analysis results of mechanical allodynia using the CIA animal mouse model. After intravenous injection of saline (n=5, 6), naïve MSCs (n=5), Edu MSCs (n=5, 6), AuS-TA (n=6), Edu-MSCs+AuS-TA (n=6), or Edu-MSCs/Aus-TA (n=6), mechanical allodynia was assessed in the CIA animal model at the indicated time points;

FIG. 11c shows a graph illustrating the analysis results of mechanical allodynia using the CIA animal mouse model. Mechanical allodynia was assessed after injection of saline (n=3, 5), Edu-MSC+AuS-TA (n=4, 5), or Edu-MSC/AuS-TA (n=4, 5), with or without laser irradiation. Laser irradiation was performed once a day for 3 days. Basal thresholds to mechanical stimulation were measured before collagen injection. Black and red arrows indicate the time of drug administration and laser irradiation, respectively;

FIG. 11d shows images (top) and a graph (bottom) illustrating analysis results of mechanical allodynia using the CIA animal mouse model. Immediately after the allodynia test, facial images were acquired and the Grimace scale according to five characteristics was analyzed. Post-injection refers to the second day after administration;

FIG. 11e shows graphs illustrating the results of an open field test using an animal model mouse. During the open field test session, the distance traveled was automatically measured on day 3 after administration of saline (n=5) or Edu-MSCs AuS-TA (n=5) and the locomotor activity was analyzed. Representative trace images are shown. The data represent the mean±standard error of the mean. *p<0.0332, **p<0.021, ***p<0.0002, ****p<0.0001;

FIG. 12a shows a graph illustrating the results of toxicological tests on mice as an experimental animal. DBA/1 mice were sacrificed at week 8 after receiving 8 injections of saline and Edu-MSCs. No significant changes were observed;

FIG. 12b shows a heatmap of the expression levels of tumorigenicity-related genes in naïve MSCs and educated MSCs (Edu-MSCs). The migration ability of ADMSCs was restored by re-education (culturing educated MSCs in stem cell medium for 24 hours) after cessation of education;

FIG. 12c shows microscopic images (top) and a graph (bottom) illustrating the analysis results of the migration ability of ADMSCs of the present disclosure. Re-educated ADMSCs showed greater enhancement of migration toward stimulated FLS compared to naïve ADMSCs and AMSCs upon cessation of training. The Data represent mean±SEM (n=3). ns<0.1234 and ****p<0.0001;

FIG. 13a shows a schematic diagram and micrographic images of PEGylated AuS (AuS-PEG) and AuS-PEG. Transmission electron micrographic (TEM) images of PEGylated AuS (AuS-PEG) and AuS PEG determined using energy-dispersive X-ray spectroscopy mapping show the distribution of Au (yellow), S (elements of PEG and cysteamine, red), but F (element of TA, blue) is not present;

FIG. 13b shows a graph illustrating the analysis results of the optimization of AuS-TA synthesis conditions;

FIG. 13c shows graphs illustrating the analysis results of the calibration curve used to determine the concentrations of AuS and TA;

FIG. 13d shows a graph illustrating the average hydrodynamic size and potential analysis results of AuS-PEG, TA, AuS-TA, and AuS-CD90-TA. The tested drugs had a size of less than 150 nm and all had negative surface charges;

FIG. 13e shows a graph illustrating the analysis results of hydrodynamic size distribution of AuS-PEG, AuS-TA, and AuS-CD90-TA;

FIG. 13f shows additional TEM and scanning-TEM (STEM) images of AuS-PEG. A black scale bar=50 nm and a white scale bar=10 nm;

FIG. 13g shows additional TEM and scanning-TEM (STEM) images of AuS-PEG. A black scale bar=50 nm and a white scale bar=10 nm;

FIG. 14a shows schematic diagrams and graphs illustrating the analysis results of FACS of Edu-MSC and Edu-MSC-AuS-TA for confirmation of drug binding. The binding of AuS-TA to Edu-MSCs was confirmed by surface-bound AuS-TA (+Alexa 488 AuS-TA for anti-CD90 antibodies and +Alexa 488 secondary antibodies);

FIG. 14b shows schematic diagrams illustrating the effect of CD90 binding on MSC apoptosis and a graph illustrating the analysis results of the survival rate. The apoptosis of MSCs was not significantly induced by the CD90 binding method for up to 72 hours (80% or more of viability);

FIG. 14c shows a schematic diagram illustrating the migration ability of Edu-MSCs and Edu-MSC-AuS-TA toward stimulated FLSs and stimulated J774, and a graph illustrating the analysis results of the migration ability. The binding of AuS-TA on the surface of Edu MSCs did not affect their migration ability toward FLS and J774. The data represent mean±SEM (n=3). ns<0.1234;

FIG. 15a shows a schematic diagram and confocal micrographic images illustrating the analysis results of endocytosis pathway for AuS-TA in RA patient-derived FLS and OA patient-derived FLS (RA FLS and OA FLS). FLS were stimulated by TNF-α at 20 ng/mL. The endocytosis pathway was inhibited by 20 μM CPZ (clathrin-mediated endocytosis), 200 μM GEN (caveolin-mediated endocytosis), and 25 μM EIPA (macrophage action);

FIG. 15b shows a schematic diagram and confocal micrographic images illustrating the analysis results of endocytosis pathway for Edu-MSCs-AuS-TA of RA FLS and OA FLS;

FIG. 16a shows a schematic diagram illustrating the preparation of a CIA mouse model according to the administration of Edu-MSCs-AuS-TA;

FIG. 16b shows a schematic diagram illustrating the administration period of Edu-MSCs-AuS-TA for the preparation of a CIA mouse model. Arthritis was induced in 6-week-old DBA/1 male mice by two injections of collagen. 28 Days after the first injection of collagen, the CIA model with moderate arthritis (arthritis severity score <4) showed a significant reduction in the incidence of osteoarthritis using Edu-MSCs-AuS-TA via the intraperitoneal route;

FIG. 16c shows representative images of the forepaws of experimental mice following the administration of Edu-MSCs-AuS-TA. The treatment was performed with saline, Edu-MSC, AuS-TA, Edu-MSC+AuS-TA (a mixture), and Edu-MSC-AuS TA (a conjugate);

FIG. 17a shows a schematic diagram illustrating the preparation of a CIA mouse model following the administration of Edu-MSCs-AuS-TA;

FIG. 17b shows a schematic diagram illustrating the administration period of Edu-MSCs-AuS-TA via PTT for the preparation of CIA mouse model. Arthritis was induced in 6-week-old DBA/1 male mice by two injections of collagen. On day 42, after the first collagen injection, the CIA model with advanced arthritis (arthritis severity score >8) was treated with NIR laser via the intraperitoneal route to the inflamed joints, followed by administration of Edu-MSCs-AuS-TA;

FIG. 17c shows images illustrating the results of hematoxylin-eosin staining and safranin-O staining of the ankle joints of the mice treated with saline+laser, Edu-MSCs+AuS-TA (a mixture), a mixture+laser, Edu-MSCs AuS-TA (a conjugate), and a conjugate+laser;

FIG. 17d shows graphs illustrating the analysis results of bone volume/tissue volume (BV/TV) and bone mineral density (BMD) using micro-CT images. The data represent mean±SEM (n=3). *p<0.0332 and **p<0.021;

FIG. 18a shows a schematic diagram illustrating the effects of photothermal therapy (PTT) on FLS and macrophages (J774);

FIG. 18b shows graphs illustrating the analysis results of the effect of PTT (AuS-TA+laser) on macrophages (J774). The transcript levels of IL22R and CD23 were normalized to the level of a GAPDH transcript. The data represent the mean±SEM of three independent experiments (n=3);

FIG. 18c shows graphs illustrating the analysis results of the effect of PTT (AuS-TA+laser) on mouse FLS. The transcript levels of IL22R and CD23 were normalized to the level of a GAPDH transcript. The data represent the mean±SEM of three independent experiments (n=3). ns<0.1234;

FIG. 18c shows graphs illustrating the analysis results of the effect of PTT (AuS-TA+laser) on mouse FLS. The transcript levels of IL22R and CD23 were normalized to the level of a GAPDH transcript. The data represent the mean±SEM of three independent experiments (n=3). ns<0.1234;

FIG. 19a shows a schematic diagram illustrating the distribution of M1 and M2 in experimental mice treated with various drugs;

FIG. 19b shows the entire image of CD86 (an M1 macrophage marker) and Dectin-1 (an M2 macrophage marker) of the ankle joints of experimental mice. The distribution of M1 and M2 at the same joint location was analyzed. The white box indicates the location of the analyzed site in FIG. 10;

FIG. 20a shows a schematic diagram illustrating an experiment performed using micro-CT scanning of the hindpaws of the CIA mouse model;

FIG. 20b shows micro-CT images illustrating the hindpaws of the CIA mouse model treated with saline and saline+laser;

FIG. 20c shows micro-CT images illustrating the hindpaws of the CIA mouse model treated with a mixture, a mixture+laser, a conjugate, and a conjugate +laser;

FIG. 21 shows graphs illustrating the results of toxicological tests on DBA/1 mice. DBA/1 mice were sacrificed 8 weeks after saline+laser, Edu-MSCs+AuS-TA (a mixture), a mixture+laser, Edu-MSCs-AuS-TA (a conjugate), and a conjugate+laser injections. No notable changes were observed in the general immunotoxicity profile of all groups compared to the untreated control group; and

FIG. 22 shows a schematic diagram illustrating the inflammation-inhibitory effect of an inflammation attenuating complex using infrared-responsive thermal stem cells of the present disclosure.

### DETAILED DESCRIPTION OF EMBODIMENTS

### Definition of Terms

As used herein, the term “rheumatoid arthritis” refers to a systemic, chronic inflammatory disease in which symptoms mainly appear symmetrically in the movable joints, and is known to be an autoimmune disease caused by an abnormality in the immune system, the cause of which is not yet clearly known. The disease is characterized by persistent inflammatory synovitis causing cartilage destruction and bone erosion, resulting in structural deformities of the surrounding joints. Symptoms associated with rheumatoid arthritis include joint swelling, joint tenderness, inflammation, morning stiffness, and especially pain which occurs when bending a joint. Subjects in advanced stages of arthritis will experience structural damage, including joint destruction, along with bone erosion (Firestein, G. S., Nature 423:356-361, 2003). Additionally, patients may have other clinical manifestations, such as damage to various organs, including skin, kidneys, heart, lungs, central nervous system, and eyes, due to vasculitis associated with the autoimmune procedure.

As used herein, the term “osteoarthritis” refers to a degenerative arthritis that occurs due to degenerative changes in the cartilage and surrounding bones in synovial joints. That is, osteoarthritis is a disease characterized by hypertrophy of the bones located below the cartilage, bone formation at the joint margin, and non-specific synovial inflammation, along with a gradual loss of articular cartilage. Osteoarthritis is a disease that occurs when cartilage is damaged due to aging or excessive physical stress (e.g., obesity, trauma, etc.). Therefore, osteoarthritis causes severe pain and movement disorders in joints that bear a large weight, such as the knee joint and hip joint, and if left untreated for a long period of time, it can even lead to joint deformation.

As used herein, the term “drug delivery carrier” refers to a material which is used to deliver a drug to a lesion where it is required and to maintain the drug for an appropriate period of time, and a method of effectively delivering a drug to a lesion using a drug delivery carrier is called a drug delivery system.

### DETAILED DESCRIPTION OF THE INVENTION

According to one aspect of the present disclosure, there is provided a stem cell-gold nanoparticle complex is provided, in which gold nanoparticles (AuNPs) loaded with an anti-inflammatory agent are bound to surface of the stem cells.

The stem cell-gold nanoparticle complex may be characterized in that a near-infrared (NIR) laser irradiation is used in combination as a photothermal therapy, and the NIR laser irradiation may be irradiated with an 800 to 900 nm NIR laser for 3 to 10 minutes. In the gold nanoparticles, PEG may be coated on the surface and steroidal anti-inflammatory drugs may be loaded by a non-covalent bond, and the weight ratio of the steroidal anti-inflammatory drugs to the gold nanoparticles may be 2:1 to 5:1.

In the stem cell-gold nanoparticle complex, the steroidal anti-inflammatory drug may be triamcinolone, hydrocortisone, prednisolone, betamethasone, or dexamethasone; the stem cell may be an embryonic stem cell, mesenchymal stem cell, or an induced-pluripotent stem cell; and the stem cell may be an embryonic stem cell, a mesenchymal stem cell, or an induced-pluripotent stem cell; and the mesenchymal stem cell may be a bone marrow-derived stem cell, a cord blood-derived stem cell, an adipose-derived stem cell, a dental pulp-derived stem cell, or a peripheral blood-derived stem cell.

In the stem cell-gold nanoparticle complex, the stem cell may be a stem cell educated as a lesion-derived cell, and the educated stem cells may be a stem cell cultured by allowing the lesion-derived cell to come in contact with a culture medium in which the lesion-derived cell is cultured.

According to another aspect of the present disclosure, there is provided a drug delivery carrier comprising the stem cell-gold nanoparticle complex as an active ingredient.

According to still another aspect of the present disclosure, there is provided a pharmaceutical composition for treating arthritis comprising the stem cell-gold nanoparticle complex as an active ingredient.

In the pharmaceutical composition, the arthritis may be rheumatoid arthritis or osteoarthritis, and the arthritis may be advanced arthritis with an arthritis severity score of 6 to 8 or higher.

In the pharmaceutical composition, the repolarization from M1 macrophages to M2 macrophages may be induced.

According to still another aspect of the present disclosure, there is provided a composition for relieving pain caused by joint inflammation, the composition including the stem cell-gold nanoparticle complex as an active ingredient.

The pharmaceutical composition of the present disclosure may vary depending on the type of patient's affected area, application area, number of treatments, treatment time, formulation, patient's conditions, type of excipients, etc. The dose is not particularly limited, but may be 0.01 μg/kg/day to 10 mg/kg/day. The daily dose may be administered once a day, or divided into 2-3 times a day at appropriate intervals, or intermittently at intervals of several days.

In the pharmaceutical composition of the present disclosure, the compound may be administered orally or parenterally, and preferably, it may be administered parenterally via intravenous injection, subcutaneous injection, intracerebroventricular injection, intracerebrospinal fluid injection, intramuscular injection, intraperitoneal injection, etc.

The pharmaceutical composition of the present disclosure may further include appropriate carriers, excipients, and diluents commonly used in the preparation of pharmaceutical compositions. Additionally, additives for a solid or liquid formulation may be used in the preparation of pharmaceutical compositions. The additives for formulations may be either organic or inorganic. Examples of excipients may include lactose, sucrose, white sugar, glucose, corn starch, starch, talc, sorbitol, crystalline cellulose, dextrin, kaolin, calcium carbonate, silicon dioxide, etc. Examples of binders may include polyvinyl alcohol, polyvinyl ether, ethyl cellulose, methyl cellulose, gum arabic, tragacanth, gelatin, shellac, hydroxypropyl cellulose, hydroxypropyl methyl cellulose, calcium citrate, dextrin, pectin, etc. Examples of glidants may include magnesium stearate, talc, polyethylene glycol, silica, hydrogenated vegetable oil, etc. As coloring agents, any of those which are normally approved for addition to pharmaceuticals may be used. Their tablets and granules may be appropriately coated with sugar, gelatin, or other appropriate coatings as needed. Additionally, preservatives, antioxidants, etc. may be added as needed. Additionally, when the pharmaceutical composition is a drug, it may further include one or more selected from fillers, anticoagulants, lubricants, wetting agents, flavoring agents, emulsifiers, and preservatives. Meanwhile, the formulation of the pharmaceutical composition of the present disclosure may be in a desirable form depending on the method of use, and it is preferable that the composition is to formulated by adopting a method known in the art so as to provide rapid, sustained or delayed release of the active ingredients, especially after the administration to a mammal. Examples of specific formulations may be any one selected from the group consisting of plasters, granules, lotions, liniments, lemonades, powders, syrups, liquids and solutions, aerosols, extracts, elixirs, fluid extracts, emulsions, suspensions, decoctions, infusions, tablets, suppositories, injections, spirits, cataplasmas, capsules, troches, tinctures, pastes, pills, and soft or hard gelatin capsules.

The pharmaceutical composition of the present disclosure may further include ingredients commonly used in the pharmaceutical composition, and may include, for example, common auxiliary agents such as stabilizers, solubilizers, and flavoring agents, and carriers.

The pharmaceutical composition or joint injection may include various carriers suitable for direct injection into the affected area.

The pharmaceutically acceptable carriers included in the pharmaceutical composition of the present disclosure are those commonly used in preparing formulations, and may include lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, etc., but are not limited thereto. The pharmaceutical composition of the present disclosure may further include, in addition to the above-described ingredients, a lubricant, a wetting agent, a sweetening agent, a flavoring agent, an emulsifier, a suspending agent, a preservative, etc. Suitable pharmaceutically acceptable carriers and formulations are described in detail in Remington's Pharmaceutical Sciences (19th Ed., 1995).

In an aspect of the present invention, there is provided a method of treating arthritis in a subject in need of comprising administrating therapeutically effective amount of the stem cell-gold nanoparticle complex to the subject.

In the method, the arthritis may be rheumatoid arthritis or osteoarthritis.

In an aspect of the present invention, there is provided a method of relieving pain caused by joint inflammation in a subject comprising administrating therapeutically effective amount of the stem cell-gold nanoparticle complex to the subject.

The term “therapeutically effective amount” used herein means the amount of a compound or a material such as a complex that, when administered to a patient for treating a disease, is sufficient to effect such treatment for the disease. The “therapeutically effective amount” will vary depending on the compound, the disease and its severity and the age, weight, etc., of the patient to be treated.

Arthritis is a chronic disease that reduces the quality of life, and although various methods have been adopted to relieve pain and inflammation and slow disease progression, there is no ideal treatment strategy for OA and RA. A variety of treatment options are clinically available, including conventional drugs such as steroids and DMARDs that aim to suppress specific cells or cytokines (J. Mucke et al., Ther. Adv. Musculoskelet. Dis. 14: 1759720X221076211, 2022). Achieving clinical absence of inflammation and disease activity and managing long-term absence of disease activity to prevent relapse are the ultimate goals of RA treatment in the clinical setting (R. M. Shammas et al., Curr. Rheumatol. Rep. 12:355-362, 2010). The European Union of Rheumatology Associations (EULAR) recently updated its recommendations for the management of RA (M. Fornaro et al., Eur. J. Clin. Invest. 51: e13363, 2021). Specifically, for newly diagnosed RA patients, the guidelines recommend that initial treatment include methotrexate, either alone or in combination with another DMARD, to achieve no or low disease activity, and that treatment be adjusted if any improvement is not observed even after 3 months (P. H. de Jong et al., Ann. Rheum. Dis. 73:1331-1339, 2014). An unmet medical need in RA still exists for non-responders, who fail after 3 months and are defined as patients with high disease activity (HDA). Likewise, as of now, more effective and specific therapeutic interventions are necessary for patients who are not allowed disease-modifying OA drugs, and are at higher risk for disease-progression. In order to alleviate arthritis in HDA patients, it is urgent to develop drugs that effectively promote tissue remodeling and repair in advanced stages of arthritis. The tissue remodeling process is not mediated by a single effector, but rather by a combined regulation of multiple factors to maintain homeostasis. Meanwhile, mesenchymal stem cells (MSCs) are considered a promising therapeutic vector for the treatment of arthritis due to their high anti-inflammatory response ability, ease of isolation, etc. Importantly, these MSCs exhibit unique tropism toward inflammatory mediators. Therefore, MSCs are being developed for targeted therapies against pathophysiological sites such as inflamed joints in OA and RA, and are currently in clinical trials. Additionally, MSCs have been shown to be effective in the treatment of arthritis through the secretion of immune-modulating molecules, including indoleamine 2,3-dioxygenase (IDO), prostaglandin E2 (PGE2), transforming growth factor beta (TGF-β), and interleukin-10 (IL-10), vascular endothelial growth factor (VEGF), and epidermal growth factor (EGF) (J. Freitag et al., Musculoskelet. Disord. 17:230, 2016). However, there are various challenges to the clinical use of MSCs as a treatment vector for arthritis, and one of which is the lack of anti-inflammatory efficacy and limited ability to migrate to sites of inflammation in vivo. Intravenously administered MSCs selectively accumulate in the lungs and liver, indicating that target tissues, such as inflamed joints, do not have sufficient concentrations of MSCs. Additionally, macrophages in the lungs, liver, and spleen typically remove administered MSCs, and MSCs transplanted into joints may be activated through anoikis (a form of programmed apoptosis resulting in detachment from the extracellular matrix (ECM)) or by increasing immune responses to host antigens may lead to apoptosis due to harsh conditions, such as low oxygen and nutrient levels (I. B. McInnes et al., N. Engl. J. Med. 365 (23): 2205-2219, 2011). Remodeling the synovial microenvironment of inflamed joints using MSCs with the aid of above-described anti-inflammatory nanomedicines is expected to be a promising therapeutic strategy for the treatment of arthritis, and has been developed into several approaches, which include genetic modification, a combination therapy with other therapies, MSC-derived vesicles or conditioned media, and pretreatment of MSCs using various compounds and inflammatory cytokines during MSC expansion or immediately prior to in vivo injection (E. M. Ghaffary and S. M. A. Froushani, Life Sci. 246:117420, 2020). Specifically, there are few preclinical studies on preconditioned MSCs, such as caffeine-pretreated MSCs, peripheral blood mononuclear cell (PBMC)-preactivated MSCs, and sIL6R-pretreated MSCs, but there have been reports on the therapeutic efficacy of MSCs for arthritis. The preconditioned MSCs did not increase migration into inflamed joints in arthritis, and most MSC-based therapies showed limited efficacy in intermediate levels of preclinical arthritis models (arthritis severity score: 0 to 4), but this was not reproduced in models of advanced arthritis (arthritis severity score: ≥8). In this regard, the conjugation of drug-loaded gold nanoparticles (AuNPs) to the surface of MSCs is a novel strategy to overcome the low anti-inflammatory efficacy of MSCs.

Gold nanoparticles (AuNPs) have shown a high promise among FDA-approved metal nanoparticles and have emerged in the past decade through biomedical applications, such as drug delivery systems, imaging probes, and therapeutics including thermal ablation. Due to their highly stable crystal forms, the AuNPs have low degradability, little corrosivity, and low reactivity with active biomolecules (N. Feliu et al., Chem. Soc. Rev, 45 (9): 2440-2457, 2016). Additionally, the strong bond between gold and sulfur ligands enables the conjugation of sulfur compounds (e.g., drugs, peptides, antibodies, or polymers) on the AuNP surface for drug delivery and specific targeting of tissues. In particular, it responds to near-infrared (NIR) lasers due to its plasmonic properties and wide absorption wavelength window (A. Guglielmelli et al., Advanced Photonics Research. 2 (8): 2000198, 2021). Photoactivation of AuNPs inside tissues with tissue transparency windows (e.g., NIR) may not only promote improved bone preservation but also induce nanoparticle heating (photothermal therapy, PTT), which inhibits inflamed synovial invasion, cartilage erosion, and expression of inflammatory cytokines in arthritis models. The PTT has received considerable attention as an adjuvant approach for the treatment of various diseases including rheumatoid arthritis and cancer. The combination of the PTT with therapeutic drugs and photosensitizers has emerged as a powerful strategy to significantly enhance the efficacy of drug-based interventions. This combined approach could be a focal point for ongoing research and analysis by resolving various medical issues.

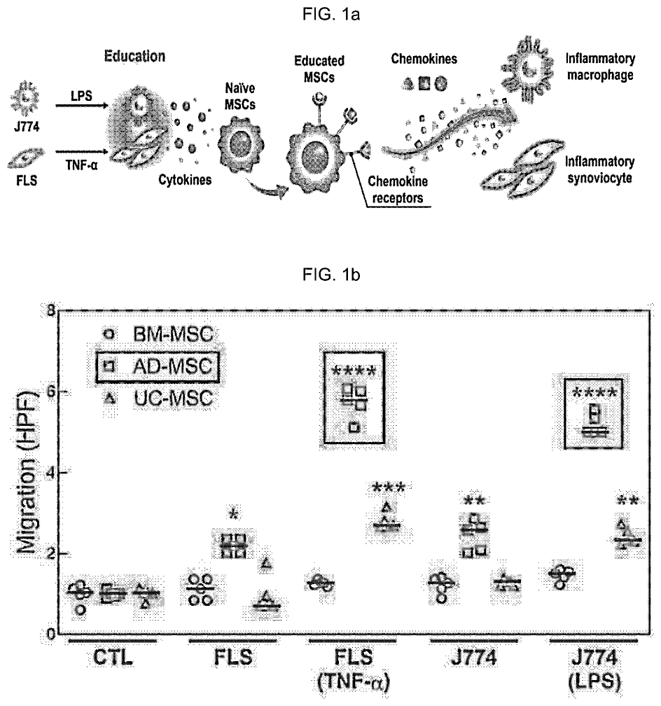

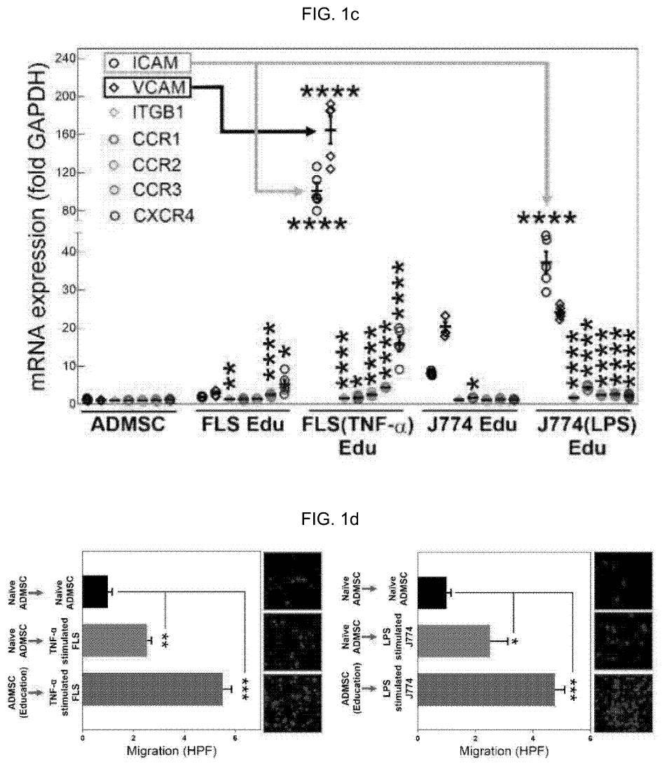

Recent studies have explored innovative strategies to resolve the challenge of precisely targeting and effectively treating inflammation in diseases such as rheumatoid arthritis (RA) (M. Zhang et al., Mater. Today Bio 14:100223. 2022). In a previous study, a strategy associated with the intravenous administration of dextran sulfate-based nanomedicines to target circulating monocytes in vivo was implemented. The nanomedicines used were internalized by monocytes and subsequently translocated to arthritic joints (C. Feng et al., Acta Biomaterialia 150: 324-336, 2022). Another approach utilized microparticles that were absorbed by MSCs in vitro and then injected retro-orbitally. This method successfully targeted locally-induced inflammation, such as in the ear, and prolonged the survival of mice with systemic inflammation. However, the methods for internal loading drugs into mesenchymal stem cells may have limitations in terms of drug delivery efficiency compared to the methods for binding drugs to the surface of mesenchymal stem cells. Previous studies have shown that internal drug-loading methods are associated with relatively low drug-loading efficiency and reduced migration ability of MSCs (S. W. Kim et al., Adv. Sci. (Weinh) 5:1700860, 2018). Therefore, the present inventors have developed a conjugate in which drug-loaded gold nanoparticles (AuNPs) are combined on the surface of the MSCs. The present inventors have developed an innovative approach to utilize an inflammation-mediated co-culture procedure defined as “education”. Importantly, the procedure does not involve genetic modification and MSCs may precisely target inflammation. Additionally, the procedure effectively controls even severe inflammation by efficiently delivering anti-inflammatory drugs to the inflamed area. This method effectively improves the target delivery of drugs to the site of inflammation, which was a limitation encountered in previous studies, and particularly, the education regimen significantly enhances the targeting of MSCs to inflamed joints, as demonstrated in the collagen-induced arthritis (CIA) model, which is an established preclinical model that closely reflects the inflammatory characteristics of RA. Star-shaped AuNPs (AuStar, AuS, and gold nanostar) were conjugated with clinical-grade triamcinolone (TA), which is a glucocorticoid used to treat various inflammatory conditions of the body, to create AuS-TA as an adjuvant drug, and AuS-TA, which is conjugated with anti-CD90 antibody along with CD90 (i.e., a membrane protein widely distributed in MSCs), was developed. Importantly, the educated MSCs (Edu-MSCs-AuS-TA), which were conjugated with the AuS-TA, were not only effective in the moderate arthritis stage, but also effectively attenuated the advanced stage of arthritis with the aid of additional NIR laser irradiation for PTT. Additionally, the Edu-MSCs-AuS-TA significantly promoted cartilage regeneration through repolarization of macrophages from the M1 (pro-inflammatory) phenotype to the M2 (anti-inflammatory) phenotype and inhibited neutrophil recruitment. Additionally, the educated MSCs promoted pain relief and increased general activity in the moderate stage of an arthritis model, and Edu-MSCs-AuS TA including PTT induced pain relief in the advanced stage of an arthritis model for the first time. In particular, the anti-inflammatory mechanism of PTT was first revealed through the present disclosure, and the downregulated expression of interleukin (IL) 22 receptor (IL22RA1), which is associated with arthritis pathogenesis, was detected for the first time in T lymphocytes together with PTT. The data obtained above suggest that the migration ability of MSCs may be enhanced without genetic modification, thereby resolving the safety-related issues. Educated MSCs actively delivered AuS-TA with a sustained photothermal effect, thereby enhancing an anti-inflammatory effect and promoting tissue remodeling in both moderate and advanced arthritis (FIG. 1).

In the present disclosure, the education procedure of mesenchymal stem cells mediated by inflammation improved their ability to migrate to inflamed joints and enhanced their anti-inflammatory effect by modulating several secreted factors (simultaneously upregulated the expression of anti-inflammatory genes and downregulated the expression of pro-inflammatory genes). Non-genetically modified MSCs (Edu MSCs) for arthritis treatment is a safe and effective strategy for inflammatory diseases including RA and OA. The developed Edu-MSCs-AuS-TA not only prevented the progression of arthritis in a moderate arthritis model, but also reversed the progression of arthritis when used in combination with PTT. Edu-MSC-AuS-TA effectively induced an anti-inflammatory synovial microenvironment (macrophage repolarization and inhibition of neutrophil recruitment), cartilage regeneration, and reduced bone erosion. Moreover, the use of Edu-MCS/AuS-TA together with PTT successfully attenuated the progression of arthritis, which could not be controlled by Edu-MSCs-AuS-TA alone. Additionally, pain relief and improved general activity were detected in both CIA models with moderate and advanced arthritis. The lack of efficacy of separately delivered Edu-MSCs and AuS TA (Edu-MSC+AuS-TA) was due to the low dose of AuS-TA in the target tissue (no targeting ability). Edu-MSCs and AuS-TA were delivered systemically via intraperitoneal injection (i.p.), and Edu-MSCs alone did not maintain anti-inflammatory efficacy in inflamed joints. Importantly, the mutual synergistic effect between Edu-MSCs and membrane-bound AuS-TA improved tissue remodeling and repair through targeted delivery of AuS-TA by Edu-MSCs toward inflamed joints. The side effects of systemically delivered drugs were reduced, and the anti-inflammatory synovial microenvironment was modulated using highly localized TAs (glucocorticoids, GCs). The present inventors predicted that the AuS-TA including PTT would further maximize the anti-inflammatory activity of Edu-MSCs by assisting to modulate the immune response of the tissue in inflamed joints. This implies that the downregulation of IL22R by PTT in T cells and FLS inhibits the differentiation of naïve CD4 T cells into Th17, and then the MSCs exhibit an anti-inflammatory effect. Therefore, even in advanced stage of arthritis, GCs promote cartilage regeneration by regulating various factors for tissue remodeling and repairing. Additionally, the use of GCs to alleviate arthritis is clinically more effective and safer than other DMARDs (P. H. de Jong et al., Ann, Rheum, Dis. 73:1331-1339. 2014). Safety issues associated with the use of GCs may be resolved using low-dose GCs in combination with nanoparticles and a cell-based vector that induce active drug delivery to target tissues compared to other tissues. The present disclosure of Edu-MSCs-AuS-TA is an example of how the combination of nanoparticles and a cell-based vector can effectively and safely help treat arthritis. Although the enhanced migration ability of MSCs after education requires further investigation, the developed procedure represents a novel approach to enhance their migration ability toward inflammatory cells without raising safety issues due to genetic modification. Additionally, Edu-MSCs-AuS-TA offers the possibility of successful treatment for HDA patients to achieve the alleviation of the disease.

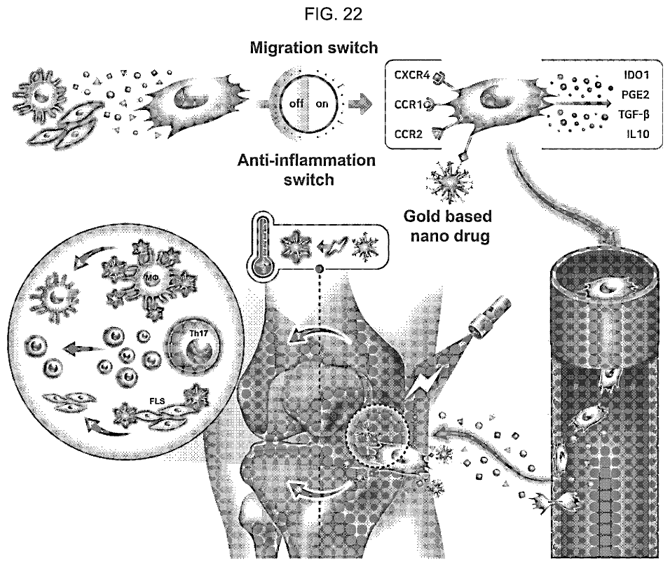

In summary, the efficacy of current clinical treatments for osteoarthritis and rheumatoid arthritis is clearly limited. Mesenchymal stem cells (MSCs) are considered a promising source of regenerative therapies, but their clinical utility is limited due to low drug efficacy and unpredictable side effects of unmodified or genetically engineered MSCs injected in vivo, respectively. In the present disclosure, a strategy to enhance the migration efficacy of MSCs into inflamed joints through an inflammatory mediator-education procedure is demonstrated. In order to enhance the limited anti-inflammatory activity of MSCs, triamcinolone-loaded gold nano-stars were conjugated to MSCs. Additionally, near-infrared laser-assisted photothermal therapy (PTT) induced by gold nano-stars significantly enhanced the anti-inflammatory efficacy of the developed drug in a model of advanced arthritis. In the present disclosure, the immunological regulatory mechanism of PTT was proposed for the first time. The expression of interleukin 22 receptors, which are associated with the pathogenesis of arthritis, were downregulated in T lymphocytes by PTT, and Th17 differentiation from naïve CD4 T cells was inhibited. Inflammatory-targeting MSCs conjugated with triamcinolone-loaded gold nano-stars promoted macrophage repolarization and reduced neutrophil recruitment in the joints. The Edu-MSCs-AuS-TA of the present disclosure significantly alleviated arthritis-associated pain, improved overall motor activity, and more importantly, induced cartilage regeneration even in a model of severe arthritis. The above results suggest that the stem cell-derived inflammation targeting complex of the present disclosure may be usefully used as a drug delivery carrier for effective drugs against a lesion by increasing the targeting ability to the corresponding lesion (FIG. 22).

Hereinafter, the present disclosure is explained in more detail through examples. However, the present disclosure is not limited to the examples disclosed hereinbelow and may be implemented in various different forms, and the following examples are provided to ensure that the present disclosure is complete and to fully inform a person of ordinary skill in the art of the scope of the invention.

### Materials and Methods

### Cell Culture

Human ADMSCs (CEFO-ADMSC, CEFO), BMMSCs (CEFO-BMMSC, CEFO), and UCMSCs (CEFO-UCMSC, CEFO) used in the present disclosure were cultured in human MSC growth medium (CEFOgro-MSC, CEFO). Human FLSs were isolated from synovial tissue of RA patients (age range: 32-59 years) using enzymatic dispersion (P. Zafari et al., Rev. Assoc. Med. Bras. 67:1654-1658, 2021). An informed consent was obtained from all patients, and the present disclosure was approved by the Eulji University Human Subjects Research Ethics Committee (Korea). The FLSs were maintained in Dulbecco's modified Eagle's medium (DMEM; 11965-092, Gibco) including 10% fetal bovine serum (FBS; 26140, Gibco) and 1% antibiotics (10378016, Gibco). Mouse T lymphocytes were isolated from mouse spleen cells using a mouse T cell isolation kit (19851, STEMCELL), and maintained in a Roswell Park Memorial Institute (RPMI 1640) medium (11875 093, Gibco) supplemented with 10% fetal bovine serum (26140, Gibco) and 1% antibiotics (10378016, Gibco). The mouse macrophage cell line J774A.1 (TIB-67, ATCC) was maintained in DMEM (11965-092, Gibco) including 10% FBS (26140, Gibco) and 1% antibiotics (10378016, Gibco), and all cultures of the present disclosure were maintained at 37° C., 5% CO2.

### Inflammation-Mediated Education Procedure

J774A.1 cells (1×10⁶) were stimulated with 50 ng/mL LPS (L5293, Sigma) in 10 mL DMEM supplemented with 10% FBS and 1% antibiotics, and CM of J774A.1 was harvested after 24 hours of culture. Human FLSs (5×10⁵) were stimulated with 20 ng/mL recombinant human TNF-α (210-TA, R&D Systems) in 10 mL DMEM supplemented with 10% FBS and 1% antibiotics, and CM of the FLSs was harvested after 24 hours of culture. Then, CM debris and cells were removed by centrifugation at 400×g at 4° C. for 5 minutes. The CMs from LPS-stimulated J774A.1 cells and TNF-α-stimulated FLSs were mixed in a 1:1 ratio in an inflammatory medium to mimic the environment of an inflamed joint. Naïve MSCs were treated with 10 mL of the inflammatory medium for 24 hours. The criterion for evaluating the reproducibility of education was the migration efficiency of the educated MSCs. The migration efficiency of Edu-MSCs into FLS in vitro met the specific criteria of an average cell number per high-power field (HPF) exceeding 70 cells in an area of 300 mm² based on five random HPF numbers per well.

### In Vitro Migration Assay

The migration abilities of ADMSCs, BMMSCs, and UCMSCs were analyzed using a transwell migration assay. One day before the assay, all of MSC types, FLSs, and J774 cells were seeded (1×10⁵) in 24-well plates (CLS3527, Corning) and cultured overnight in 500 μL complete medium. In order to stimulate FLS and J774 cells, TNF-α or LPS was added for additional 24 hours. All types of MSCs (1×10⁵) were seeded on the top of Transwell chambers (#3422, polycarbonate membrane, 24-well format, 8-μm pore size, Corning). After 6 hours, cells spread on the membrane surface were fixed with 100% methanol for 1 minute and stained with 4′,6-diamidino-2 phenylindole (DAPI) (D1306, Molecular Probes). 10 Fields per sample were examined at 400× magnification (high-power fields, HPF). The number of MSCs migrating through the membrane pores toward MSCs (control group), FLSs, or J774 cells was counted, and the mean cell number/HPF was determined.

### Isolation and Culture of FLS and In Vitro Analysis

The isolation and culture of FLS and in vitro analysis of primary OA and RA FLSs were isolated from synovial tissue donated by a 68-year-old female patient with osteoarthritis at Nowon Eulji Medical Center of Eulji University (Korea), and as described above, it was donated from a 44-year-old female patient with rheumatoid arthritis through enzymatic dispersion. The tissues were then cultured in a monolayer, and prior consent was obtained from all patients, and were approved by the Human Subjects Research Ethics Committee of Eulji University (Approval IRB Number: 2022-10-011-005) and Kyungpook National University Hospital (Approval IRB Number: 2052-040903). FLSs were cultured in Dulbecco's modified Eagle's medium including 1% penicillin-streptomycin and 10% non-therapeutic FBS under the conditions of 5% CO2 and 37° C. In the present disclosure, FLSs of 3 to 7 passages were used. Moreover, 20 ng/mL of TNF-α was treated to stimulate FLSs. The drug was treated at 500 ng/mL based on triamcinolone, and the naïve MSCs and the average number of treated Edu-MSCs were 1×10⁴, which was the same as Edu-MSC-AuS-TA.

### Flow Cytometry

In order to detect AuS-TA-bound ADMSCs, AuS-TA was synthesized by non-covalently conjugating Alexa Fluor™ 488 conjugate (S11223, Invitrogen) using a previously described method (J. Y. Park et al., ACS Appl. Mater. Interfaces 12 (58): 38936-38949, 2020). In order to estimate the neutrophil population in the total cells of the synovial tissue, anti-mouse CD11b (M1/70, BioLegend) and Ly-6G (also known as GR-1; 1A8, BioLegend) were used for FACS analysis. In summary, cells were acquired on a BD LSRII flow cytometer (BD Biosciences) and all FACS data were analyzed using FlowJo software (ver10.1, FlowJo, LLC). In order to investigate the apoptosis of Edu-MSCs induced by AuS-TA binding, Edu-MSCs AuS-TA (5×10⁵) were cultured in 10 cm dishes for 72 hours. The apoptosis of Edu-MSCs was monitored every 24 hours by additional incubation with Annexin V (1:100, A35122, Molecular Probes) at room temperature for 30 minutes and analyzed using FACS.

### In Vivo Fluorescence Imaging

In order to investigate the enhanced targeting ability of ADMSCs after inflammation-mediated education, ADMSCs were labeled with Vivo Track 680 (2819777, PerkinElmer) to track cell migration. Mice were injected three times over 3 days with 1×10⁶ Vivo Track 680-labeled ADMSCs, and in vivo fluorescence images (excitation: 676 nm, emission: 696 nm) were collected in a Spectral Instruments Imaging System (AMI HT). In vivo fluorescence imaging data sets were acquired and quantified using AURA imaging software (ver4.0, Spectral Instruments Imaging System).

### RT-qPCR

In order to confirm the expression of chemokine receptors and cytokines, qPCR was performed using a real-time PCR detection system (CFX 384, Bio-Rad) according to the manufacturer's protocol. Specifically, total RNA was isolated from ADMSCs and FLSs using the QIAzol Lysis reagent (79306, QIAGEN). The isolated RNA was used to synthesize cDNA using M-MLV reverse transcriptase (M1701, Promega) under the conditions of 45° C. for 60 minutes and 95° C. for 5 minutes. The changes in mRNA levels were determined using qPCR. 4 μL (200 ng) of cDNA, 1 μL (20 pM) each of the forward and reverse primer solutions, 12.5 μL of FastStart Universal SYBR Green Master (4913850001, Roche), and 6.5 μL of distilled water (DW) were mixed to a final volume of 25 μL and used for amplification. Cycle threshold (Ct) values were calculated using the CFX Real-Time PCR Detection System software (Bio-Rad). Relative fold changes in gene expression were calculated according to the comparative Ct method (2-ΔCt model) and then normalized to the mean expression of GAPDH. The base sequence information of the primers used for the amplification is summarized in Table 1 below.

TABLE 1
Primer Sequence Information

| | | SEQ |
|---|---|---|
| | | ID |
| Primer | Sequence (5′->3′) | NOS: |
| GAPDH F | TGCTGAGTATGTCGTGGAGT | 1 |
| GAPDH R | AGATGATGACCCTTTTGGCTC | 2 |
| IL-1β F | GCATGGTATGGACTGTGGAC | 3 |
| IL-1β R | GCAATATCCTCTGGGTCCTG | 4 |
| TNF-α F | TGTCTACTGAACTTCGGGGT | 5 |
| TNF-α R | GAGGGTCTGGGCCATAGAA | 6 |
| IL-6 F | CTTCACAAGTCGGAGGCT | 7 |
| | TAAT | |
| IL-6 R | AGTCGATCATCGTTGTTCA | 8 |
| | TAC | |
| Arg-1 F | GCAGAGGTCCAGAAGAATGG | 9 |
| Arg-1 R | ACACATAGGTCAGGGTGGAC | 10 |
| IL-4 F | AGATGGATGTGCCAAACGTC | 11 |
| IL-4 R | AATATGCGAAGCACCTTGGA | 12 |
| IL-10 F | GTCATCGATTTCTCCCCTGTG | 13 |
| IL-10 R | GTAGACACCTTGGTCTTGGAG | 14 |
| IL-22 | TACGTGTGCCGAGTGAAGAC | 15 |
| receptor F | | |
| IL-22 | GCCCAGATAACAGAGCAAGC | 16 |
| receptor R | | |
| CD-23 F | CACAGCCTCCGATTCTCTAG | 17 |
| CD-23 R | TGGAGCCCTTGCCAAAATAG | 18 |

### RNA Sequencing

In case of control and test RNA, libraries were constructed using the QuantSeq 3′ mRNA-Seq Library Prep Kit (Lexogen, Inc., Austria) according to the manufacturer's instructions. Briefly, 500 ng of total RNA was prepared, and reverse transcription was performed after hybridizing the RNA with an oligo-dT primer including an Illumina-compatible sequence at the 5′ end. After the digestion of the RNA template, a second-strand synthesis was initiated by a random primer including an Illumina-compatible linker sequence at the 5′ end. The double-stranded library was purified using magnetic beads to remove all reaction components. The library was amplified to add the full adapter sequence required for cluster generation. The final library was purified from the PCR components. A high-throughput sequencing was performed using single-end 75 sequencing using NextSeq 500 (Illumina Inc., USA). The QuantSeq 3′ mRNA-Seq readings were aligned using Bowtie2 (Langmead and Salzberg, Nat. Methods. 9:357-359, 2012). Bowtie2 indexes were generated from representative transcriptome sequences for alignment to genome assembly sequences or genome and transcriptome. The aligned files were used to assemble the transcriptome, estimate the abundance thereof, and detect differential expression of genes. Differentially expressed genes were determined based on the number of unique and multiple alignments using Bedtools (Quinlan et al., Bioinformatics 26 (6): 841-842. 2010). The reading count data were processed within R using Bioconductor (Gentleman et al., Genome Biol. 5: R80, 2004) using the quantile normalization method via EdgeR (R Development Core Team, 2016). The genes used for classification were searched based on the DAVID (//david.abcc.ncifcrf.gov/) and MEDLINE (//www.ncbi.nlm.nih.gov/) databases.

### Anti-CD90-AuS-TA Conjugate

PEGylated AuS was synthesized using a seeded growth method. A gold seed solution and citrate capped AuNPs were prepared by conventional synthetic methods (J. Y. Park et al., ACS Appl. Mater. Interfaces 12 (35): 38936-38949, 2020). Briefly, the solution was injected with vigorous stirring at all stages, and a solution of 25 mM gold (III) chloride trihydrate (HAuCl₄, Sigma) and 1 N hydrogen chloride (HCl, OCl) was added to DW. A gold seed solution, AgNO₃ (3 mM), ascorbic acid (100 mM), and O-(3-carboxypropyl)-O′-[2-(3-mercapto propionylamino)ethyl]-polyethylene glycol solution (Sigma) (5 mg/mL) were added sequentially. The reaction was performed for 2 hours, and the PEGylated AuS solution was centrifuged in a centrifugal filter tube (Millipore) at 3,000 rpm at room temperature for 10 minutes and washed several times with DW. The PEGylated AuS solution was redispersed in 2 mL of 50 mM 2-morpholinoethanesulfonic acid (MES) buffer (pH 6.0). PEGylated AuS-TA was prepared via a N-hydroxysuccinimide (NHS)-1-ethyl-3-(dimethylaminopropyl) carbodiimide hydrochloride (EDC) coupling reaction. EDC (Sigma; 15 mg/mL) and NHS (Sigma; 30 mg/mL) were added to the PEGylated AuS solution with vigorous stirring, and the mixture was stirred at room temperature for 30 minutes. The mixture was then centrifuged at 8,000 rpm for 10 minutes and dispersed in 2 mL of 10 mM PBS. The anti-CD90 antibody (ab23894, Abcam; 0.1 mg/mL) was added to the AuS-TA bound to the surface of ADMSCs with vigorous stirring overnight. A TA solution (2 mg/mL) was added to the AuS-CD90 binding solution, allowed to bind at room temperature for 4 hours, and the AuS-CD90-TA solution was collected by centrifugation at 3,000 rpm.

### Physicochemical Characterization

The hydrodynamic size distribution and electrical potential of nanomedicines including AuS-TA were evaluated using a Litesizer 500 (Anton Paar) according to the manufacturer's instructions. AuS and TA non-covalently attached to AuS were quantified by determining absorbance using UV-vis spectrophotometry (Biochrom). AuS showed a characteristic absorbance peak in the range of 825 nm, which was used to calculate the AuS concentration. The TA bound to AuS showed a peak in the range of 242 nm, which was subtracted from the initial absorbance intensity of AuS. For Edu-MSCs-AuS TA, ADMSCs showed an absorbance profile overlapping with TA (242 nm). Therefore, the TA amount of Edu-MSCs-AuS-TA was measured based on the AuS-TA ratio of AuS and TA. The concentration of each solution was extrapolated using the linear standard curve for AuS and TA. The morphology, crystallinity, and EDS mapping of AuS-PEG and AuS-TA complexes were analyzed using TEM (F30, Tecnai, USA) and STEM (JEM-ARM200F, JEOL, Japan).

### MSC Nanomedicine Conjugate

After 24 hours of ADMSCs education procedure, the synthesized AuS-TA was bound to the ADMSC surface via the anti-CD90 antibody of AuS-TA. The AuS TA (200 μg/mL) in 5 mL of serum-free medium was added to 1×10⁶ ADMSCs. After incubating for 2 hours, ADMSCs were detached from the dish using 0.25% trypsin (1 mL, Gibco) at 37° C. for 5 minutes and mechanically dissociated into a single cell suspension after the addition of FBS (10%, 0.5 mL). The separated cells were passed through a 40 mm cell strainer (93070, SPL), centrifuged, and washed with PBS. After quantifying the TA in Edu-MSCs-AuS-TA, the drug was diluted for each experiment.

### Confocal Microscopy

For the MSC analysis of nanomedicine conjugation, ADMSCs were cultured on poly-d-lysine coated coverslips. The AuS-TA labeled with Alexa 488 was conjugated to ADMSCs using the above method. Then, Edu-MSCs-AuS-TA were washed twice with PBS and fixed with 2% paraformaldehyde in the medium at 4° C. overnight. After blocking with 1% bovine serum albumin for 2 hours, the cells were incubated with goat anti-rabbit IgG H&L (Texas Red®) (ab6719, Abcam) at room temperature for 2 hours to visualize anti-CD90 antibodies on AuS-TA. In order to visualize the cell bodies of ADMSCs, rhodamine-labeled phalloidin including a mounting medium (H-1600-10, Vector Laboratories) was used for coverslipping. For histological analysis of joint tissue, target MSCs and macrophages in joint tissue were stained with primary antibodies and Alexa Fluor 594- or Alexa Fluor 488-labeled secondary antibodies. The excised joint tissues were deparaffinized and cultured with the following antibodies: human anti-TNF-α (ab6671, Abcam), human anti-CD133 (ab264538, Abcam), mouse anti-F4/80 (ab6640, Abcam), mouse anti-CD133 (ab264538, Abcam) and CD86 (ab234401, Abcam), and mouse anti-Dectin 1 (ab300497, Abcam). Then, the joint tissues were treated with the following secondary antibodies: anti-mouse IgG Alexa Fluor 594 for TNF-α (ab150116, Abcam); anti-rabbit IgG Alexa Fluor 488 for CD133, CD86, and Dectin-1 (ab150077, Abcam); and anti-rat IgG Alexa Fluor 594 for F4/80 (ab150160, Abcam). After the staining, samples were visualized using a confocal fluorescence microscope (EVOS M7000, Invitrogen), and fluorescence intensities were quantified using ImageJ and Celleste image analysis software (Invitrogen).

For the analysis of the endocytosis pathway, FLSs were stimulated with 20 ng/mL TNF-α and labeled with a Vybrant DiD cell labeling solution (V22887, Invitrogen) 2 hours before the drug treatment. The endocytosis pathway inhibitors were treated, 1 hour prior to the drug treatment, with 20 UM chlorpromazine, CPZ (clathrin-mediated endocytosis), 200 μM genistein, GEN (caveolin-mediated endocytosis), and 25 μM 5-(N-ethyl-N-isopropyl) amiloride, and EIPA (macroporosis). 500 ng/mL of Alexa 488-labeled AuS-TA and Edu-MSCs-AuS-TA (Alexa 488-labeled) were treated with triamcinolone-based FLSs. FLSs were fixed with 4% paraformaldehyde and stained with DAPI 2 hours after drug uptake. Additionally, intracellular trafficking was performed by tracking early endosomes with an anti-EEA1 antibody (ab2900, Abcam) and late endosomes with anti-M6PR (ab2733, Abcam). The drugs were treated for 6 and 12 hours, and then fixed and stained with antibodies.

### CIA Model

Male DBA/1 mice (4-6 weeks old, body weight of 20-25 g, Orient Bio, Seoul, Korea) were housed in cages for 7-14 days in a climate-controlled, specific pathogen-free environment at 22° C. with a 12/12 h light/dark cycle. All animal experiments were performed in accordance with the Gachon University guidelines for the care and use of laboratory animals. CIA was induced by subcutaneous administration of a mixture of complete Freund's adjuvant (100 μg of Mycobacterium tuberculosis, 7009, Chondrex) and 100 μg of bovine type II collagen (20021, Chondrex) to the base part of the tail, and 21 days after the first injection, a further injection was given of a mixture of incomplete Freund's adjuvant and 100 μg of bovine type II collagen. CIA animals were randomly assigned 7 days after additional vaccination.

### Arthritis Scoring Using Near-Infrared Laser and Drug Treatment

The severity of arthritis was assessed by scoring foot swelling using the following scale: 0, no signs of swelling; 1, mild inflammation and swelling of individual toes; 2, moderate inflammation and swelling of toes; 3, severe swelling of the entire foot; 4, maximum swelling of the limb (D. D. Brand et al., Nat. Protoc. 2 (5): 1269-1275, 2007). Each paw of the mouse was observed and the total arthritis index score was recorded. In order to evaluate the therapeutic efficacy of moderate arthritis, mice with an arthritis severity score of less than 4 were randomly divided into five groups (saline, Edu-MSCs, AuS-TA, Edu-MSCs+AuS-TA, and Edu-MSCs-AuS-TA) (n=5/group). In order to evaluate the therapeutic efficacy in advanced stage of arthritis, mice with an arthritis severity score exceeding 8 points were randomly divided into five groups (saline+laser, Edu-MSC+AuS-TA (a mixture), a mixture+laser, Edu-MSC-AuS-TA (a conjugate), and a conjugate+laser (n=5/group). The drug concentration varied depending on the amount of TA bound to 1×10⁶ ADMSCs (single injection). Therefore, the Edu-MSC and Edu-MSC+AuS-TA groups were injected with 1×10⁶ ADMSCs, and the AuS-TA and Edu-MSCs+AuS-TA groups were injected with the same amount of AuS-TA as in the Edu-MSCs+AuS-TA group. Cells and mice were exposed to an 808 nm NIR laser (240 mW cm-2 and 1.2 W cm-2, respectively) for 5 minutes. The changes in arthritis index in each group were recorded for 3 weeks. After 3 weeks of treatment, mice were sacrificed, and limbs were collected for histological analysis.

### 3-Dimensional Micro Computed Tomography (CT)

After all control groups and CIA models were sacrificed, the hindpaws were cut open and treated with 10% neutral buffered formalin (NBF) for 1 hour. Micro-CT was performed on the hind ankle joint using a micro-CT scanner (SkyScan 1276; SkyScan, USA) with exposure conditions of 60 kVp for 2 hours, 57 μA, 300 ms/frame with a 360-degree view, and a field of view of 68 mm.

### Modified OARSI Score

The chondroprotective activity of the drug was assessed by the modified OARSI score of cartilage sections stained with safranin O. The scoring criteria were as follows: structure (slight surface irregularity from 1 to 10, fibrillation and/or erosion into the subchondral bone), cellularity (increased or slightly decreased from 1 to 4, no cells), chondrocyte replication (from 1 to 4 at several doublets, multicellular nests), and the maximum possible score is 18 (S. S. Glasson et al., Osteoarthritis Cartilage 18: S17-S23, 2010).

### Mouse T Cell Isolation and Differentiation

After mincing the mouse spleen tissue, splenic red blood cells were removed using RBC lysis buffer (420302, BioLegend). The separated splenocytes were cultured with magnetic beads from a naïve CD4 T cell isolation kit (EasySep™ Mouse CD4+ CD62L+ T Cell Isolation Kit, STEMCELL) and separated using a magnet (EasySep™ Magnet, STEMCELL). Positively selected CD4⁺ CD62L⁺ T cells were treated with AuS-TA for 2 hours and then treated with laser for 5 minutes. After 5 hours of incubation, in order to achieve Th17 polarization, cells were stimulated, for 3 days, with IL-6 (20 ng/mL), IL-23 (10 ng/ml), IL-1B (10 ng/mL), rhTGF-β (2 ng/mL), anti-IL-4 antibody (10 μg/mL), anti-IFN-γ antibody (10 μg/mL), and anti-IL-2 antibody (10 μg/mL) (CytoBox Th17, Miltenyi Biotec).

### Behavioral Test

The temperature and illumination in the laboratory were maintained at 23±3° C. and 390 1×, respectively. Institutional Animal Care and Use Committee guidelines were followed and the minimum number of animals used for obtaining statistically significant results were used. In order to obtain concrete and repeatable behavioral data, all behavioral tests were performed between 11:00 and 17:00 by observers who were blind to group assignment. The data are expressed as mean±standard error of the mean.

### Mechanical Allodynia Test

For the allodynia test, experimental animals were acclimated to the test environment once every two days for one week. When the animals began to show a response of running away or thrashing, they were considered to be in pain. In order to measure mechanical allodynia, mice were placed in a clear plastic chamber on a high table and allowed to acclimate for about 30 minutes. The 50% withdrawal threshold was determined using the up-down method using a von Frey filament set (0.02-2 g, North Coast Medical, Morgan Hill, CA, USA).

### Facial Grimace Test

In order to determine the nature of pain in the CIA model, the facial grimace test was performed as previously described after having some modifications (D. J. Langford et al., Nat. Methods 7:447-449, 2010). The test was performed on a group of experimental subjects with allodynia. Facial images were photographed for 20 minutes immediately after the allodynia test. For each animal, four to five clear facial images were clicked at 3-5 minute intervals and the grimace scale was analyzed for behavioral units including orbital tightening, nose-cheek bulge, whisker tightening, and ear position. Each action unit was scored as 0, 1, or 2 points, and the average grimace scale score was calculated as the average score of all action units.

### Open-Field Test

The open-field test was performed according to established methods after having some modifications to assess open field test motor activity (H. Lim et al., Pain, 158, 1666. 2017). Specifically, the open-field device consisted of a four-sided 48 cm×48 cm×52 cm (L×W×H) acrylic box. Mice were placed in the center of the test chamber and allowed to explore freely for 60 minutes. The total distance traveled within the chamber during the test period was automatically measured using a video tracking system (T. D. Gould et al., Mice, Chapter 1, 2009). After each test, the test device was washed with 70% ethanol and dried for 5 minutes to remove olfactory signals.

### Safety Analysis

Peripheral blood samples were collected at the end of the study period. For the analysis of peripheral circulating blood cells, blood samples were placed in labeled vials including heparin (5 units/mL) and transported onto top of ice for hematological analysis. Blood cells were automatically counted (Sysmex F-820 Blood Counter, Toa Medical Electron). For serum analysis, blood was allowed to clot at room temperature. The clots were removed, centrifuged at 2,000×g at 4° C. for 15 minutes, and the supernatant was retained. ALT, AST, BUN, Crea, TP, and Alb levels were measured using commercial clinical chemistry reagent kits (HUMAN).

### Statistical Analysis

Statistical significance was analyzed using Student's t-test for two groups of samples and analysis of variance for three or more samples, followed by three Newman-Keuls comparison tests. Statistical significance of the p value was determined as follows: ns<0.1234, *p<0.0332, **p<0.021, ***p<0.0002, ****p<0.0001.

### Example 1: Inflammatory Mediator Education Course to Enhance Targeting of MSCs

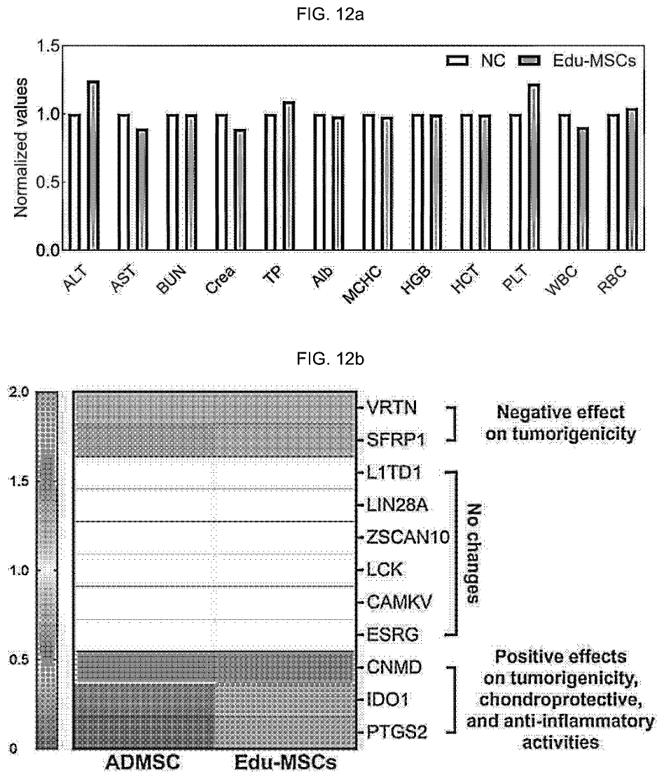

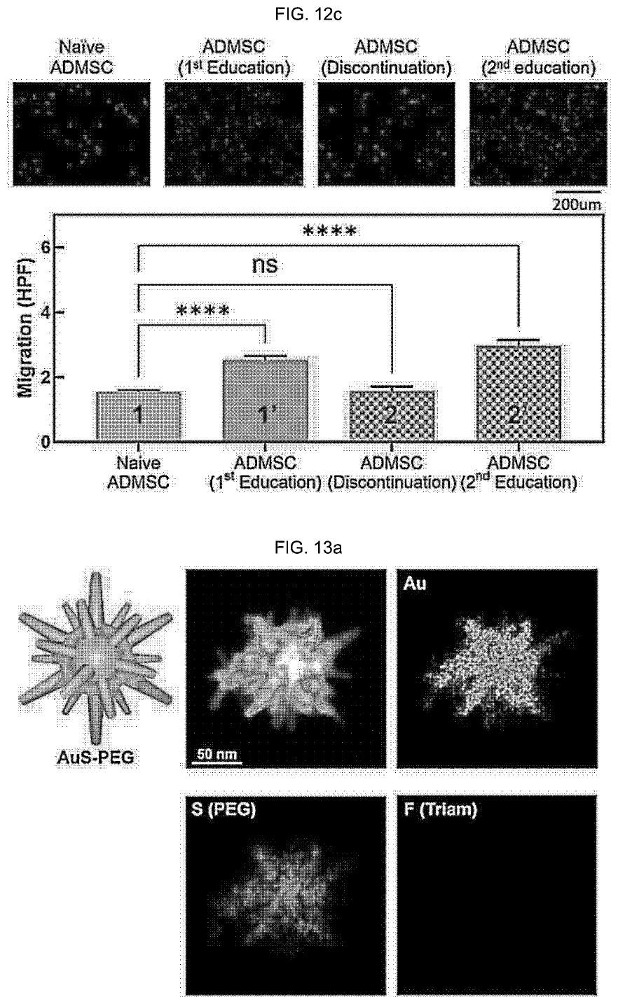

The present inventors performed in vitro inflammation-mediated education experiments to enhance the targeting ability of MSCs to inflamed FLSs and macrophages. In the present disclosure, an inflammation-mediated educational procedure was developed to enhance the migration ability of MSCs (FIG. 1a). Conditioned media (CM) was collected from lipopolysaccharide (LPS)-stimulated J774 (macrophages) and tumor necrosis factor-alpha (TNF-α)-stimulated fibroblast-like synoviocytes (FLSs) cultured for 24 hours and treated with naïve MSCs for 24 hours. In order to identify the lineage of MSCs exhibiting the best migration ability after education, early passages (1-3) of adipose tissue-derived MSCs (ADMSCs), bone marrow-derived MSCs (BMSCs), and umbilical cord-derived MSCs (UCMSCs) were analyzed using an in vitro migration assay (FIG. 1b). Among them, ADMSCs showed the highest migration ability toward FLS or J774, and the migration toward inflammatory cells (stimulated FLS or J774) was significantly enhanced in both ADMSCs and UCMSCs (FIG. 1b). ADMSCs were selected as the drug delivery vector in all experiments because of their enhanced ability to migrate toward inflammatory macrophages and FLSs. The enhanced migration ability of MSCs was assessed using an in vitro migration assay (FIG. 1d). Preconditioned MSCs treated with media including inflammatory cytokines exhibited significantly higher rates of migration into inflammatory macrophages (J774) and synoviocytes (FLS) than naïve MSCs that had not been subjected to in vitro education. Interestingly, the migration efficiency of the stem cells, which was initially reduced due to intervention of the education procedure, could be restored by subsequent retraining (FIG. 12c).

Next, the present inventors examined the mechanism of MSC education and the factors that influence and improve migration ability. Chemokines secreted by rheumatoid synovium include stromal cell-derived factor 1 (SDF 1/CXCL12), macrophage inflammatory protein-1α (MIP-1α/CCL3), and monocyte chemoattractant protein 1 (MCP-1/CCL2), and upon normal activation, T cells are expressed and secreted (RANTES/CCL5). This may attract MSCs to inflamed joints (Z. Szekanecz et al., Front. Immunol. 10:2182, 2019). After blocking the chemokine receptors of MSCs that can recognize CXCL12 (CXCR4), CCL3 (CCR1), CCL2 (CCR2), and CCL5 (CCR3) by antibodies, and the inhibitory effect of MSCs on cell migration was analyzed (FIG. 1e). The migration ability to inflammatory FLSs was significantly influenced by CCR2, CCR3, and CXCR4, whereas the migration ability to inflammatory macrophages was significantly influenced by CCR1 and CCR2. The changes in MSCs during the education were analyzed using reverse transcription-quantitative polymerase chain reaction (RT-qPCR). The expression of chemokine receptors was significantly upregulated by the education procedure using inflammatory media (FIG. 1c). The CM from inflamed FLSs induced upregulation of the expression of CCR2, CCR3, CXCR4, intercellular adhesion molecules (ICAMs), and vascular cell adhesion molecules (VCAMs) compared to the CM from naïve FLSs. ICAM and VCAM are involved in MSC tethering, which involves long, thin membrane cylinders called tethers that connect MSCs to attachment sites on blood vessels for migration. The CM from inflammatory J774 cells also significantly upregulated the expression of CCR1, CCR2, CCR3, CXCR4, ICAMs and VCAMs compared to the CM from naïve J774 cells (FIG. 1c).

### Example 2: Enhanced Targeting Ability of MSCs

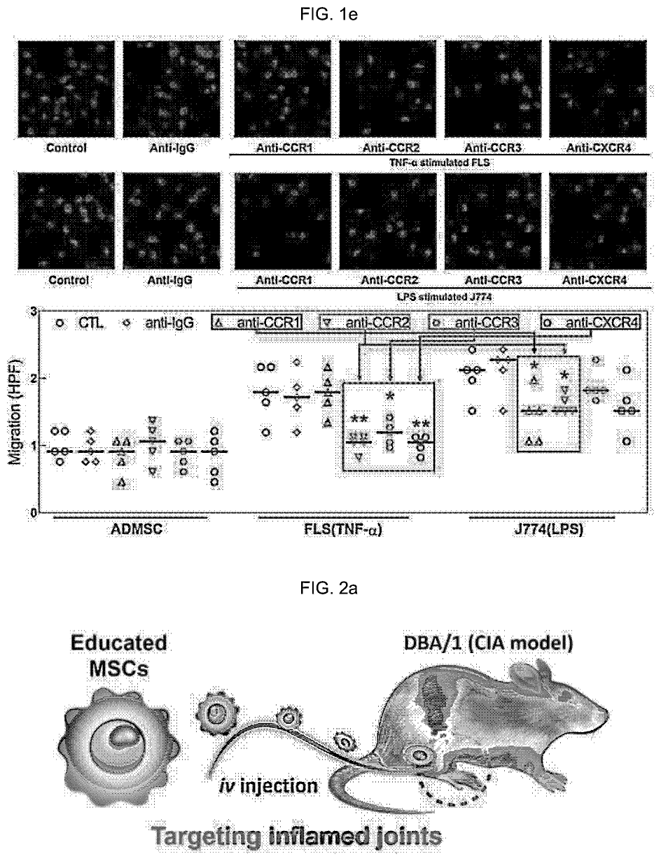

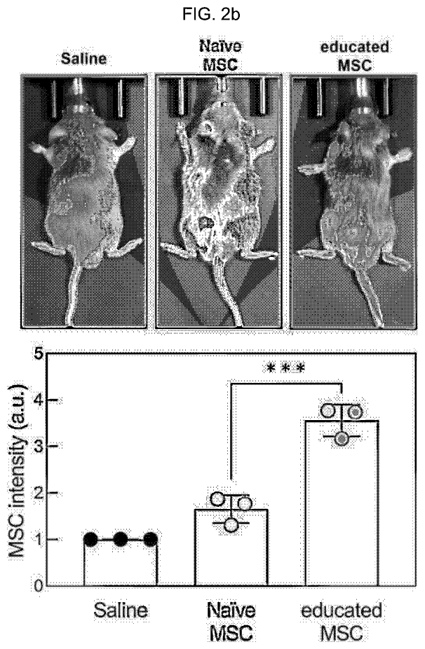

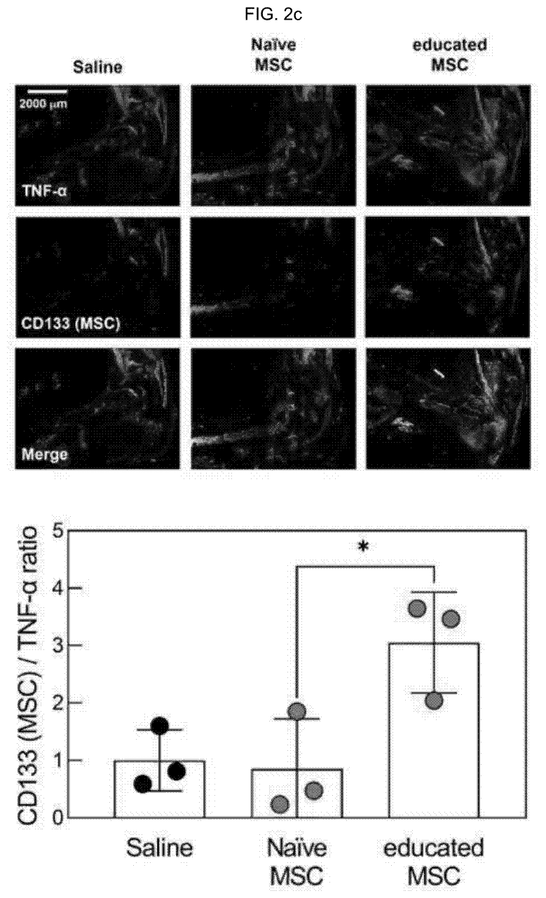

The present inventors examined the enhanced targeting ability (in vivo) of educated MSCs to inflamed joints. The enhanced migration ability of educated MSCs was evaluated in vivo using a collagen-induced arthritis (CIA) model (FIG. 2a). The educated MSCs were intravenously injected three times over a week in the CIA mouse model and tracked using an in vivo imaging system (IVIS) analysis. The educated MSCs showed significantly enhanced targeting ability to inflamed joints located in the forepaws and hindpaws as well as the tail compared to naïve MSCs (FIG. 2b). The histological analysis of the joint tissue of the hind paw confirmed a more significant migration of educated MSCs (FIG. 2c). Specifically, immunofluorescence staining revealed strong colocalization of mesenchymal stem cells stained with the major inflammatory cytokine TNF-α (red) and the stem cell marker CD133 (green). Based on the results of in vitro and in vivo targeting ability, the education procedure significantly affected the migration ability of MSCs toward inflamed joints in an arthritis model, making them an effective therapeutic vector toward inflamed joints or inflammatory sites in an arthritis model.

### Example 3: Genome-Wide Expression Profiling

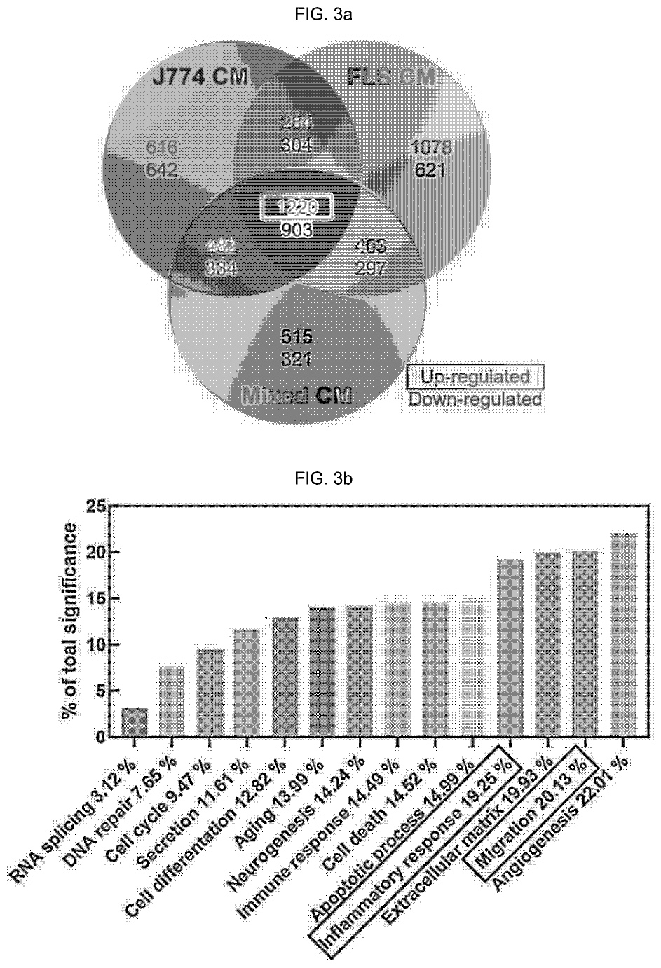

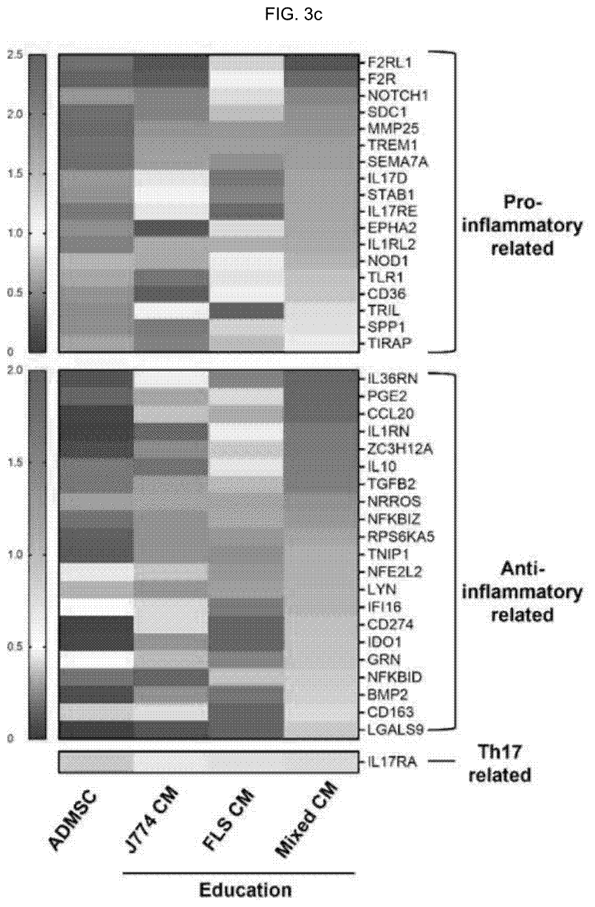

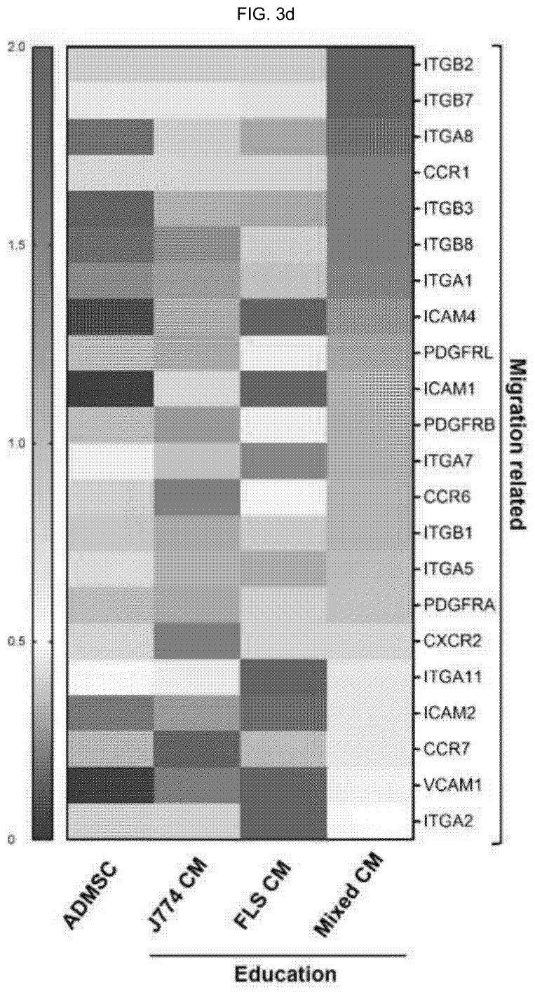

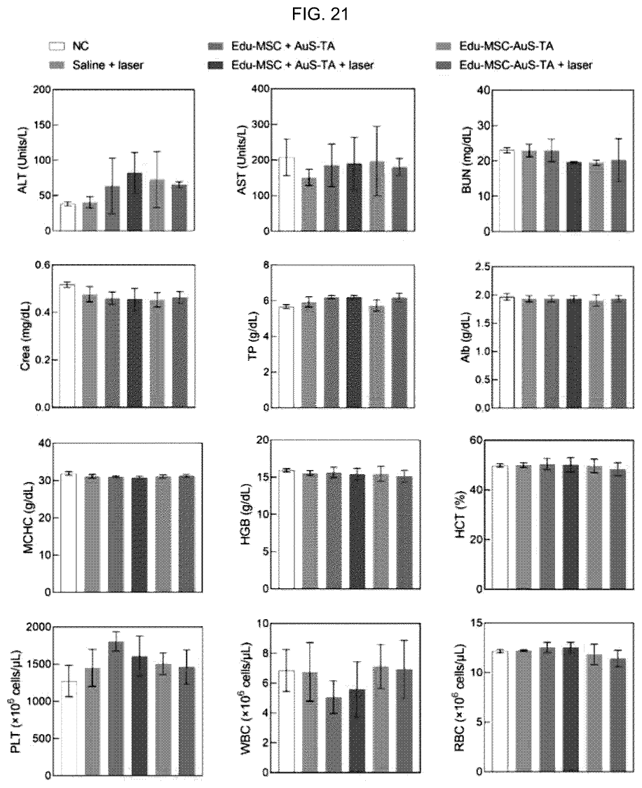

The present inventors performed RNA sequencing to detect changes in the MSC transcriptome after education. Genome-wide expression profiling was performed using MSCs treated with J774 CM and/or FLS CM. A total of 2,123 transcripts (fold change >1.25 or <0.8), 1,220 upregulated transcripts, and 903 downregulated transcripts, were deregulated in all three groups (FIG. 3a). Among the 2,123 transcripts, the expression of 18 inflammation-related genes was downregulated, and the expression of 21 anti-inflammatory genes exhibiting anti-inflammatory and chondroprotective activities, including indoleamine 2,3-dioxygenase (IDO), prostaglandin E2 (PGE2), transforming growth factor beta (TGF-β), and interleukin-10 (IL-10), was downregulated (FIG. 3c). Interestingly, IL17RA, which is an IL-17 receptor, was upregulated by MSCs during the education procedure (FIG. 3c). It is worth noting that IL-17 is primarily re-secreted by Th17 differentiation. Additionally, in order to determine whether gene expression could at least partially explain the variation in migration ability, the present inventors generated gene sets based on functional similarities using the DAVID and MEDLINE databases. Significantly increased gene sets included angiogenesis, migration, extracellular matrix and inflammatory responses (total significance %>15%) (FIG. 3b). The number and types of genes that are deregulated in the classified gene sets, which may potentially affect migration, are those associated with cell differentiation (327 upregulated, 165 downregulated), immune response (225 upregulated, 51 downregulated), migration (134 upregulated, 49 downregulated), cell cycle (75 upregulated, 52 downregulated), inflammatory response (96 upregulated, 12 downregulated), and angiogenesis (49 upregulated, 21 downregulated). Additionally, among the total of 2,123 transcripts, the present inventors analyzed upregulated genes of chemokine receptors mainly associated with migration. Twenty-two chemokine receptors, including many members of integrin family as well as the CC and CXC families of chemokine receptors, and the ICAM and VCAM families, were upregulated following the education procedure (FIG. 3d). The implications of education for MSC are multifaceted and complex, and thus further exploration is needed to elucidate the changes in functions and potential risk factors associated with educated MSCs. In particular, concerns about stem cell tumorigenicity continue to be an important matter for consideration across therapeutic applications and thus require further investigation. Although it is true that existing research in this field has a limitation, certain key parameters for assessing stem cell tumorigenicity were confirmed through rigorous investigation. These parameters include upregulation of LIN28, ESRG, CAMKV, CNMD, L1TD1, LCK, VRTN, ZSCAN10, PTGS2, and IDO1 genes and downregulation of SFRP1, which serve as essential markers for assessing tumorigenic potential. The analysis results of the present disclosure raised a controversy regarding the proposition that the education procedure may enhance the tumorigenicity of MSCs. This controversy arises due to conflicting results, particularly due to the upregulation of CNMD (associated with chondroprotective activity), IDO1 (associated with anti-inflammatory activity) and PTGS2, genes implicated in tumorigenesis, and upregulating of SFRP which suggests the presence of an opposing effect on tumor formation, and downregulating and binding of VRTN. Additionally, several genes, including LIN28A, ESRG, CAMKV, L1TD1, LCK, and ZSCAN10, remained unchanged (FIG. 12b), adding complexity to the above-raised issue. Meanwhile, chondromodulin (CNMD) plays an important role in the development and maintenance of cartilage, which is a specialized connective tissue found in joints (P. Klinger et al., Arthritis Rheum., 63 (9): 2721-2731, 2011). Additionally, indoleamine 2,3-dioxygenase 1 (IDO1) plays an anti-inflammatory role in immune regulation by suppressing T cell responses and promoting immune tolerance (M. Takamatsu et al., J. Immunol. 191 (6): 3057-3064, 2013). These unique functions of genes contribute to the complexity of assessing the tumorigenic potential of educated MSCs using mixed CMs (Edu-MSCs). Furthermore, although the in vivo toxicity assessment described in FIGS. 12a and 21 did not reveal any notable toxicity in response to multiple injections of Edu-MSCs, it is essential to further investigate the consequences of treating MSCs with inflammatory media. In conclusion, the results derived from RT-qPCR and RNA sequencing substantially support the hypothesis that the education significantly enhances the migration ability of MSCs.

### Example 4: Synthesis and Binding of Nanomedicines to Educated MSCs Membranes

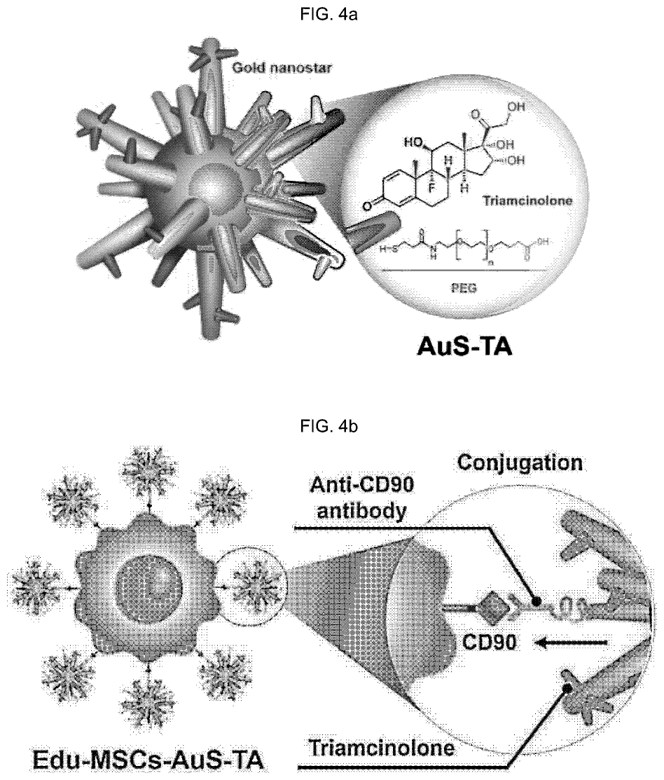

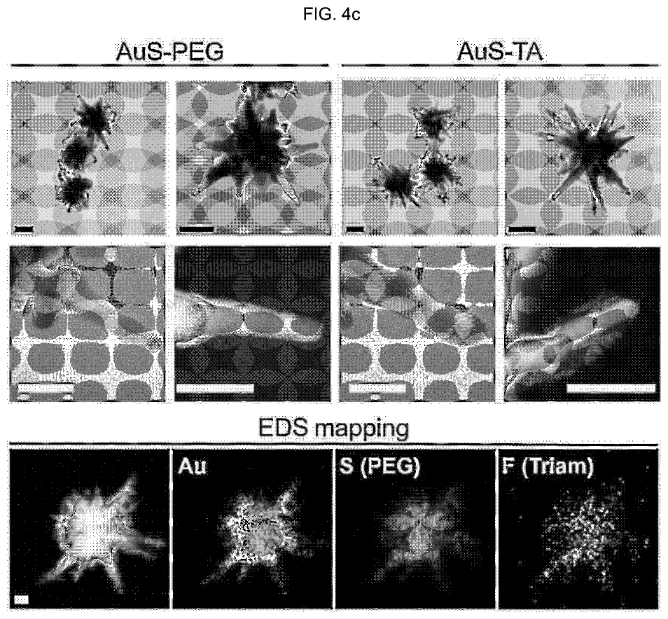

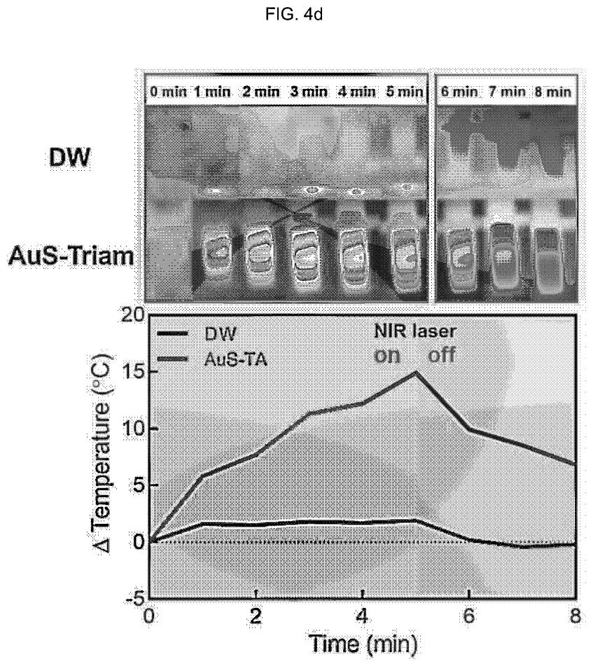

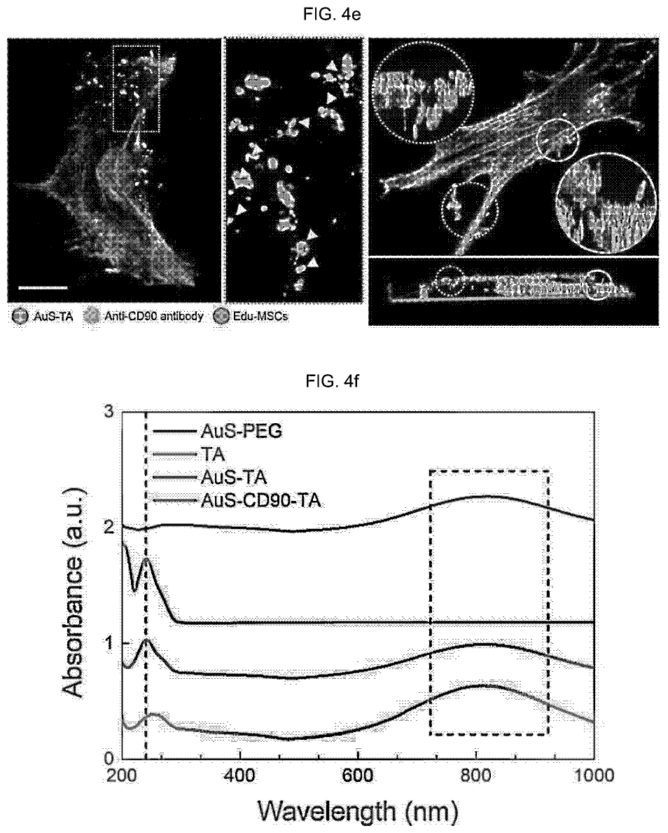

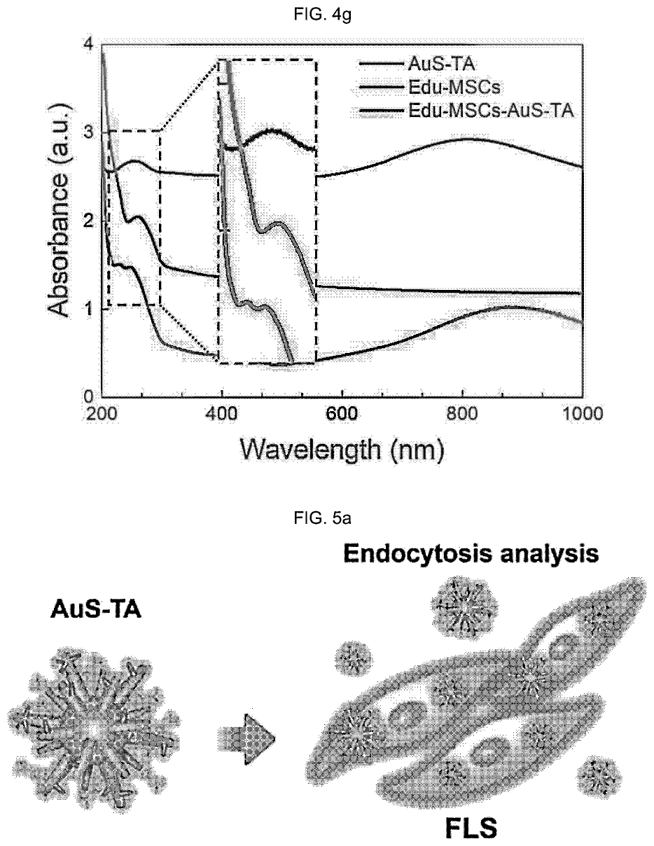

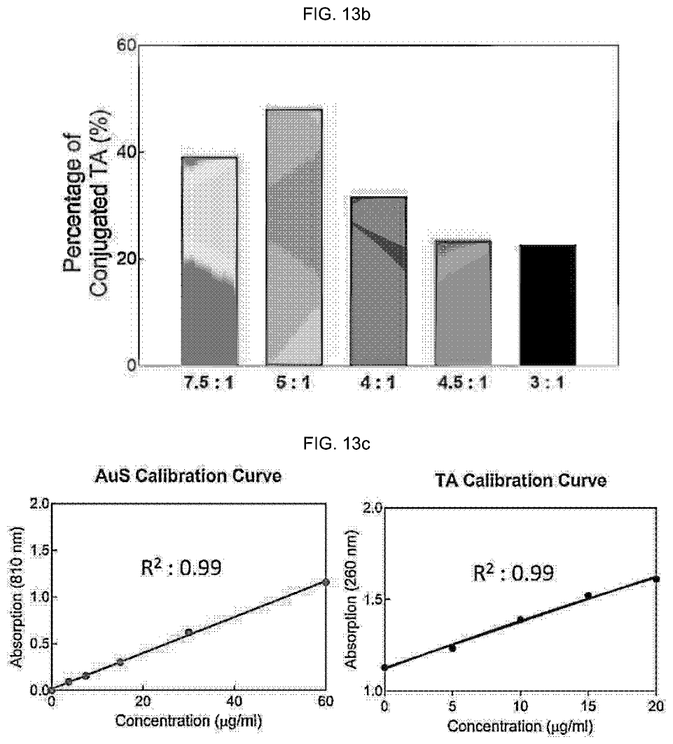

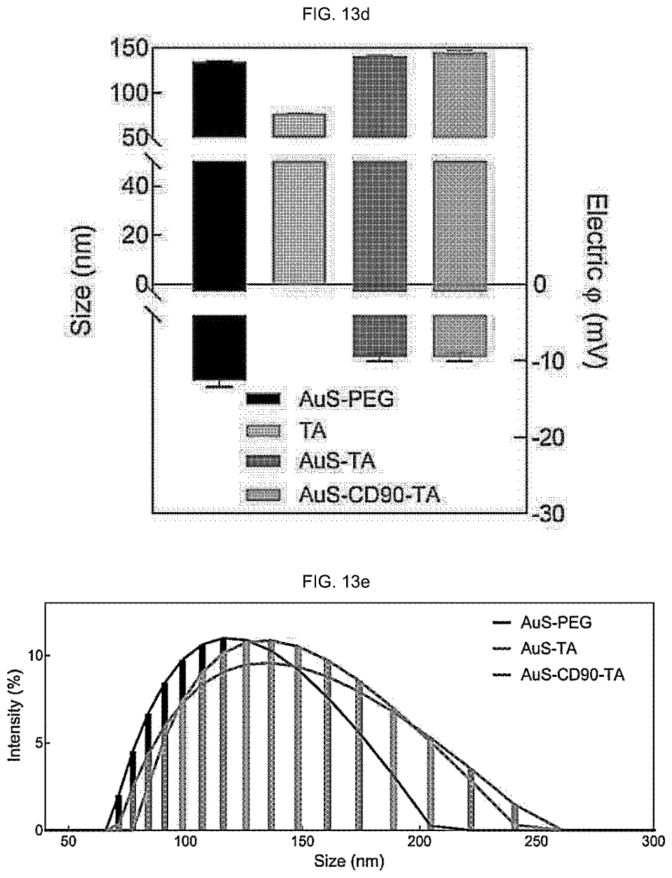

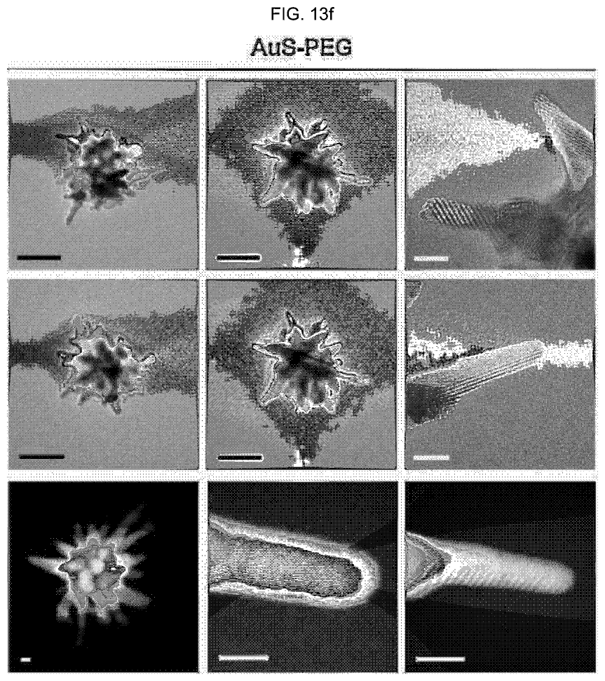

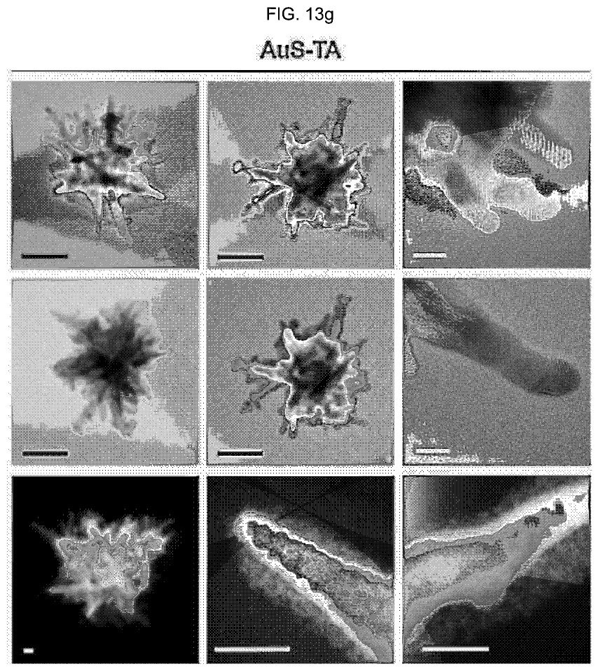

According to an embodiment of the present disclosure, PEGylated star-shaped gold nanoparticles (AuS) were conjugated with anti-CD90 antibodies (Abs) and triamcinolone (TA). The initial step involved covalent binding of AuS to anti-CD90 antibodies, and simultaneously non-covalent binding to TA was performed (FIG. 4a). After the education (Edu-MSC) to produce Edu-MSC-AuS-TA on the MSC membrane using anti-CD90 antibody in AuS-TA, the binding of AuS-TA to MSC was performed (FIG. 4b). The amount of anti-CD90 antibodies in 1 mg of AuS-TA was measured by Bradford assay. The amount of injected anti-CD90 Ab was 10 μg/mL, and when the EDC/NHS (ratio) was 1:2, it was 1.036 μg (±0.3) (Table 1). The optimization of binding efficiency of triamcinolone (TA) to AuS was determined based on the weight ratio of AuS and TA, which were added at the initial stage (Table 2 and FIG. 13b). When the weight ratio of AuS and TA was initially set to 5:1, the final weight ratio of AuS and TA reached 2:1, at which point the TA binding efficiency was observed to be highest. The total amount of TA was estimated based on the amount of AuS attached to the MSCs using the standard curve generated from absorbance measurements.

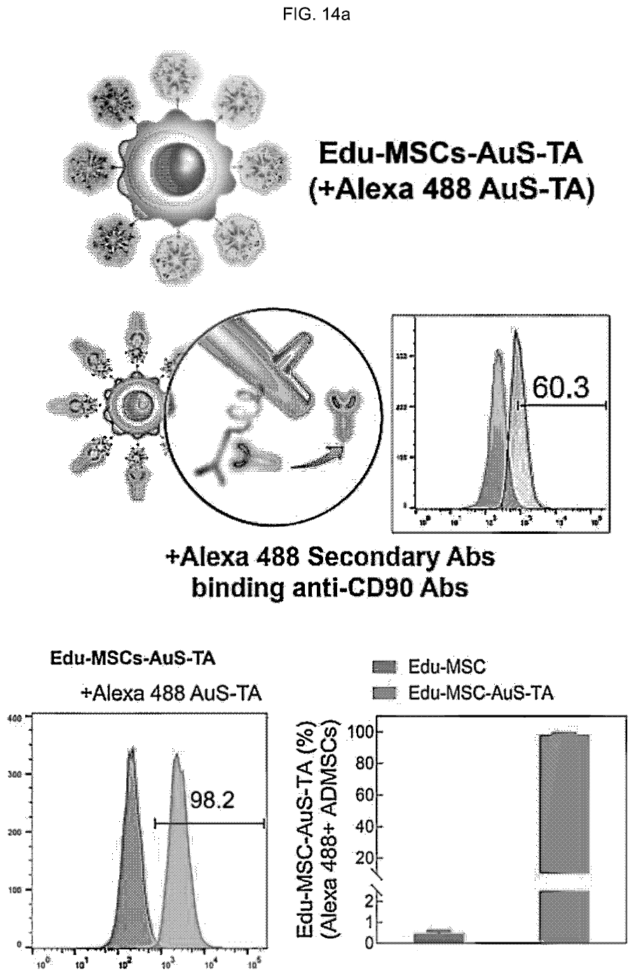

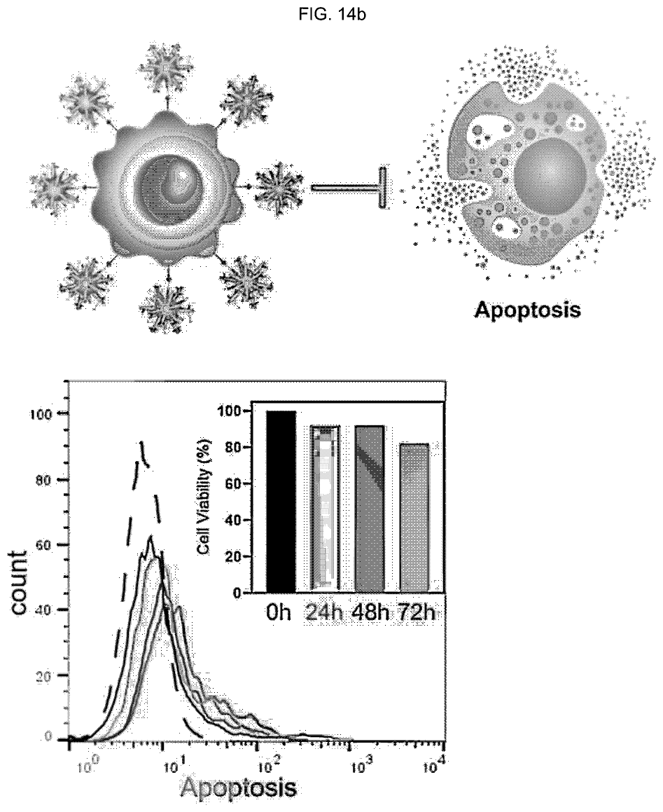

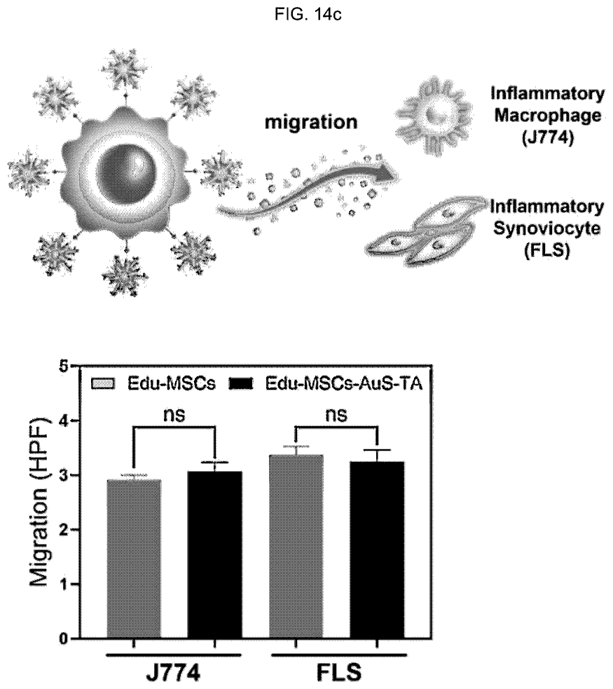

Subsequently, the amount of TA attached to AuS was calculated using the TA:AuS weight ratio derived above, and was determined to be 1:2.26 (+0.625) (Table 3). The star-shaped morphology of the PEGylated gold nanostar triamcinolone complex (AuS-TA) was confirmed using transmission electron microscopy (TEM) and scanning TEM (STEM) (FIGS. 4c and 13f, 13g). The difference in shape and structure of PEGylated AuS (AuS-PEG) and AuS-TA was confirmed as an additional structure covering the branches of AuS, and the energy-dispersive X-ray spectroscopy (EDS) showed signals of Au and S (sulfur: PEG and cysteamine), and F (fluorine: TA) at the same locations (FIG. 4c). While the PEGylated AuNPs without TA showed no signal from fluorine, the exact matching of each chemical material in AuS-TA indicates that TA was sufficiently bound to the PEGylated AuNPs (FIGS. 4c, 13a, 13f, and 13g). Additionally, the hydrodynamic sizes of AuS-PEG, free TA, AuS-TA, and AuS-TA-conjugated CD90 (AuS-CD90-TA) for MSC binding were found to be 135 nm, 77 nm, 141 nm, and 145 nm, respectively (FIGS. 13d and 13e). The surface charge was found to be close to neutral for glass TA, whereas AuS-PEG, AuS-TA, and AuS-CD90-TA were found to be negatively charged (FIG. 13d). Photoactivation of AuS-TA by NIR laser irradiation was detected using a thermal imaging infrared camera (FIG. 4d). The heating of AuS-TA by NIR laser successfully increased delta T (ΔT) to 15° C. within 5 minutes. Therefore, the NIR laser-responsive AuS-CD90-TA of MSCs was successfully identified. The confocal images of Edu-MSCs-AuS-TA also confirmed that AuS-TA was successfully bound to the Edu-MSCs membrane (FIG. 4e). Additionally, UV-visible (UV-vis) spectroscopy showed absorbance peaks at 810 nm (AuS), 260 nm (MSC), and 242 nm (TA) for AuS-TA and Edu-MSCs-AuS-TA (FIGS. 4f and 4g). In particular, the absorbance peaks of AuS-TA and Edu-MSC shifted slightly after the binding of AuS-TA to Edu-MSC (FIG. 4g). Most of the Edu-MSCs-AuS-TA showed fluorescence intensity from AuS-TA labeled with a green fluorescent protein in fluorescence-activated cell sorting (FACS) analysis (FIG. 14a). The above results suggest that Edu-MSC and AuS-TA were successfully combined under optimized conditions. Additionally, the binding of AuS-TA to Edu-MSC did not induce any notable apoptotic signal in MSCs for up to 72 hours (FIG. 14b). The amount of anti-CD90 antibodies conjugated to the AuS-PEG is summarized in Table 2 below. The amount (μg) of anti-CD90 antibodies bound to 1 mg of AuS-PEG was measured by Bradford assay (n=3, mean±SD). In addition, the results of the optimization analysis of the AuS TA synthesis conditions are summarized in Table 3 below, and the TA content and binding efficiency of the AuS-TA conjugate before and after attachment to Edu-MSC are summarized in Table 4 below.

TABLE 2
Amount (μg) of anti-CD90 antibodies conjugated to AuS-PEG

| | |
|---|---|
| Amount of anti-CD90 | EDC/NHS (ratio) |

| antibodies injected (μg/mL) | 1:1 | 1:2 |
|---|---|---|
| 2.5 | 0.342 ± 0.81 | 0.489 ± 0.84 |
| 5 | 0.921 ± 0.56 | 0.962 ± 0.23 |
| 10 | 0.983 ± 0.44 | 1.036 ± 0.30 |
| 20 | 1.022 ± 0.47 | 1.068 ± 0.61 |

TABLE 3
Optimization of AuS-TA synthesis conditions

| | Mass of | | Mass of | | |
|---|---|---|---|---|---|
| AuS-CD90-TA | AuS after | AuS | bound | Bound TA | Final |
| (Mass ratio of | binding to | Loss | TA | per AuS | ratio |
| AuS:TA) | TA (mg) | (%) | (mg) | (%) | of AuS |
| Sample 1 (7.5:1) | 2.8 | −78.46 | 1.1 | 39.2 | 2.54:1 |
| Sample 2 (5:1) | 2.7 | −73.00 | 1.42 | 52.5 | 2:1 |
| Sample 3 (4:1) | 12.43 | −68.92 | 3.94 | 31.6 | 3:1 |
| Sample 4 (4.5:1) | 12.26 | −31.89 | 2.9 | 23.6 | 4.2:1 |
| Sample 5 (3:1) | 41 | −46.34 | 9.315 | 22.7 | 4.4:1 |

TABLE 4
TA content and binding efficiency of AuS-TA conjugates

| Percentage of bound TA | Mass of bound TA per | Final ratio of TA:AuS |
|---|---|---|
| per AuS (before bound | Edu-MSC | (after Edu-MSC |
| Edu-MSC) | (1 × 10⁶) | conjugation) |
| 52.5% (±6.8) | 210 μg (±30) | 1:2.26 (±0.625) |

### Example 5: Analysis of Endocytosis Pathway of Edu-MSCs-AuS-TA

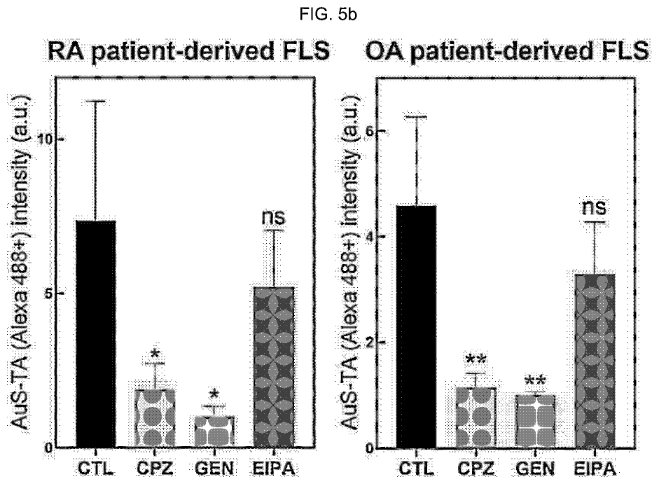

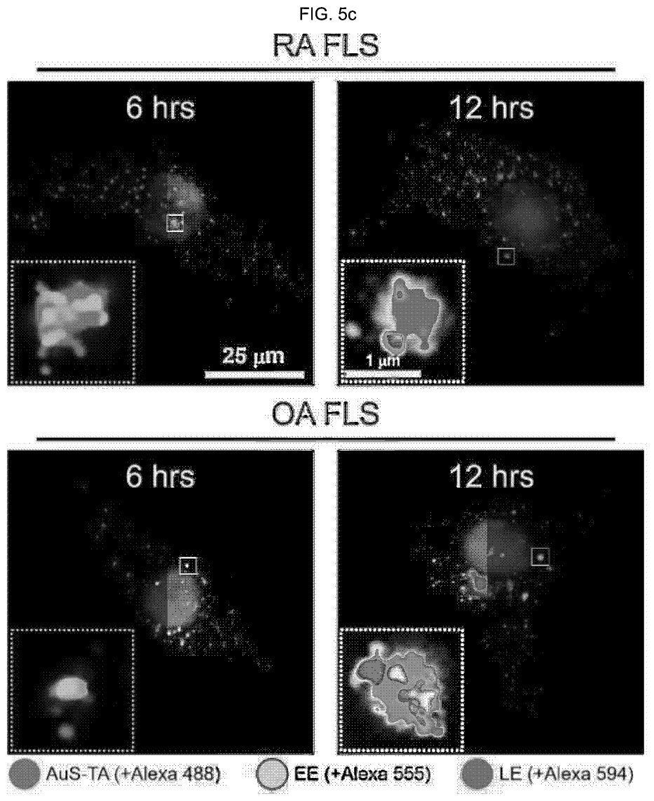

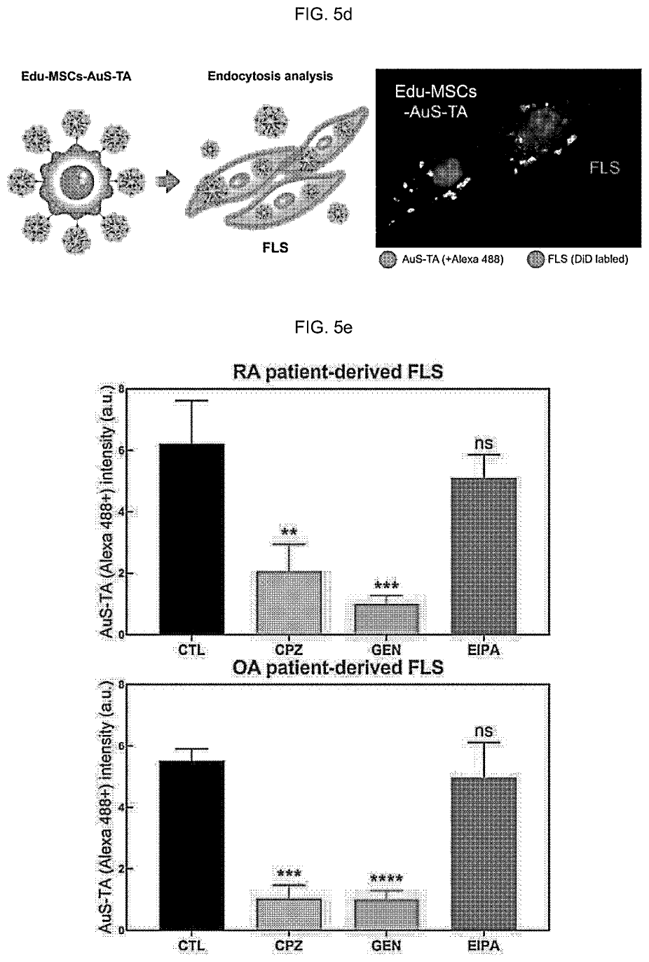

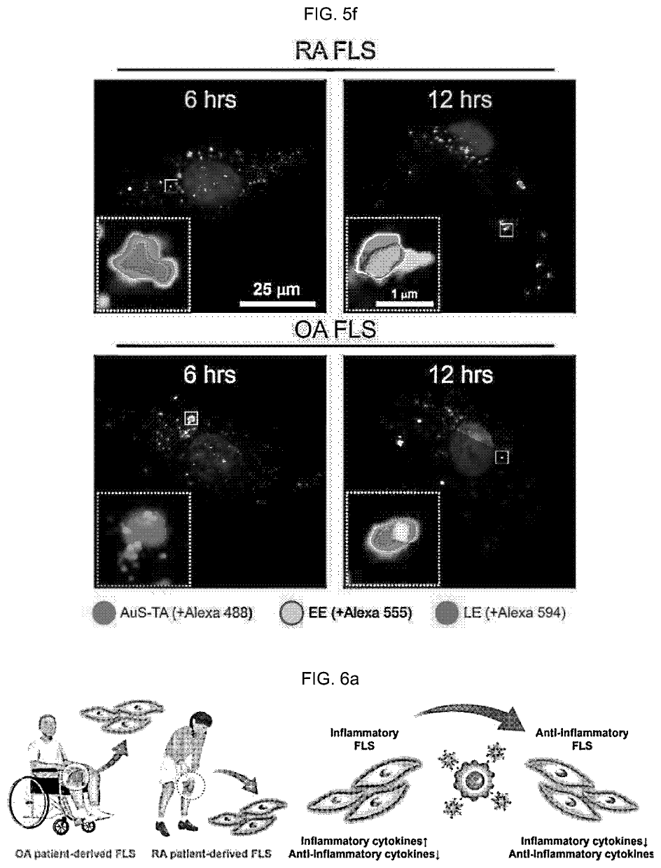

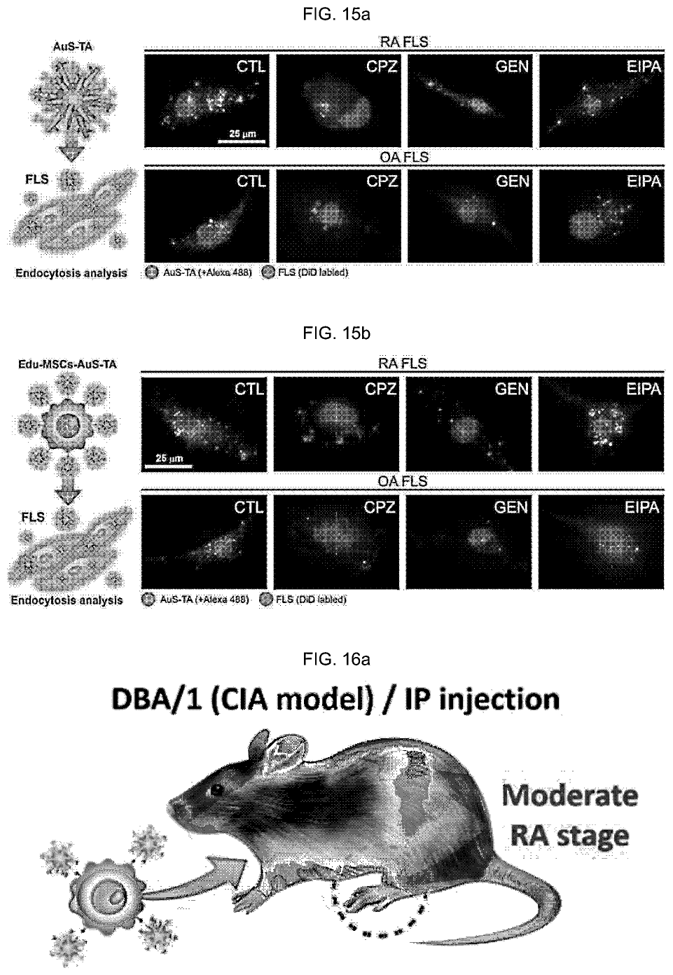

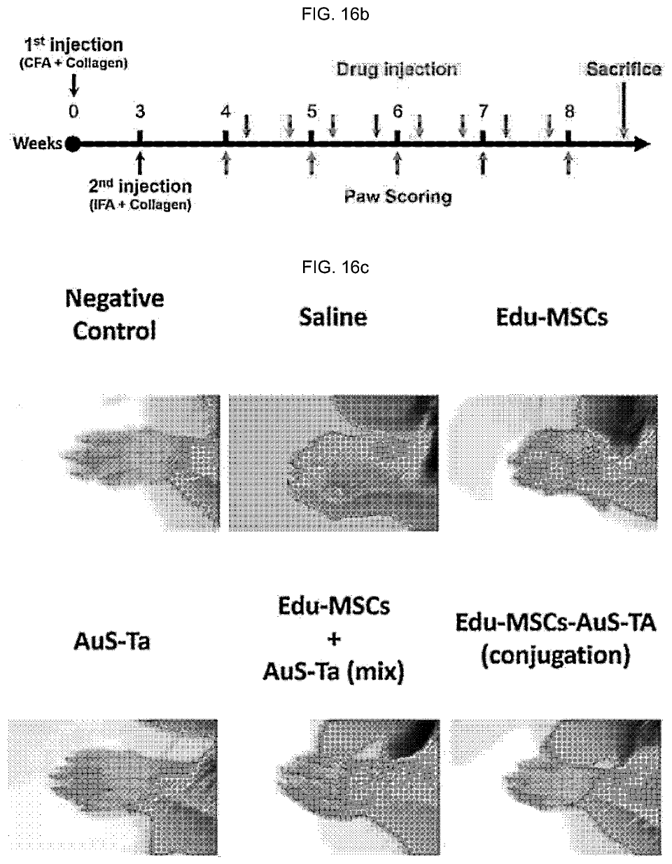

Nanoparticles are internalized into cells by endocytosis, and the different profiles of endocytosis pathways (e.g., clathrin- and caveolae-mediated endocytosis, macropinocytosis) and endosomal intracellular transport pathways (i.e., early endosomes, late endosomes, and lysosomes) are shown to vary depending on their physicochemical properties (M. J. Mitchell et al., Nat. Rev. Drug Discov. 20 (2): 101-124, 2021). In order to analyze the endocytosis profiles of AuS-TA and Edu-MSCs-AuS-TA in FLS, the endocytosis pathway and endosomal intracellular transport pathway were analyzed (FIGS. 5a and 5d). The endocytosis pathway analyses were performed using inhibitors of clathrin-mediated endocytosis (chlorpromazine, CPZ), caveolae-mediated endocytosis (genistein, GEN), and macropinocytosis (5-(N-ethyl-N-isopropyl) amiloride, EIPA) on RA and OA patient-derived FLS (RA FLS and OA FLS) (FIGS. 5b and 5e). AuS-TA or Edu-MSCs-AuS-TA (Alexa 488 labeled with AuS-TA) were absorbed for 2 hours on RA FLS and OA FLS (DiD labeled). AuS-TA was primarily absorbed by clathrin- and caveolin-mediated endocytosis, and macropinocytosis was a secondary pathway of AuS-TA endocytosis in both RA FLS and OA FLS (FIGS. 5b and 15a). The internalized AuS-TA was further analyzed by tracking to the major cellular compartments involved in endocytosis (early endosomes having EEA1 and late endosomes and lysosomes having M6PR) (FIGS. 5c and 5f). AuS-TA or Edu-MSCs-AuS-TA (Alexa 488 labeled with AuS-TA) were absorbed into RA FLS and OA FLS for 6 and 12 hours. After 6 hours, most of the internalized AuS-TA (a green-red fluorescence signal) was localized to early endosomes (a red fluorescence signal), but after 12 hours, some AuS-TA signals were also detected in late endosomes (a purple fluorescence signal) (FIG. 5c). Importantly, Edu-MSCs-AuS-TA delivered AuS-TA-bound membranes to FLS, and internalization of AuS-TA was observed in FLS (FIG. 5d). There was no significant difference in the endocytosis pathway (FIG. 5e) and intracellular transport pathway (FIGS. 5f and 15b) between AuS-TA and Edu MSCs-AuS-TA. These results clearly demonstrated for the first time that cellular nanomedicine transport was successfully achieved through caveolin and clathrin uptake and endo-lysosomal intracellular tracking in FLS.

### Example 6: Immunomodulatory Ability of Edu-MSCs-AuS-TA

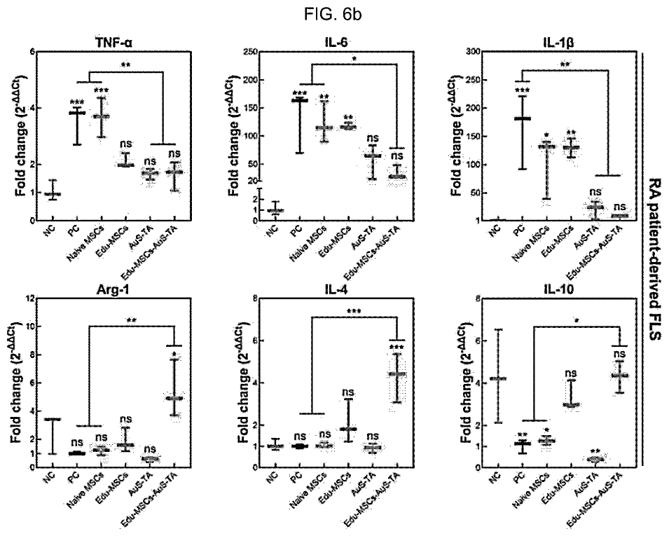

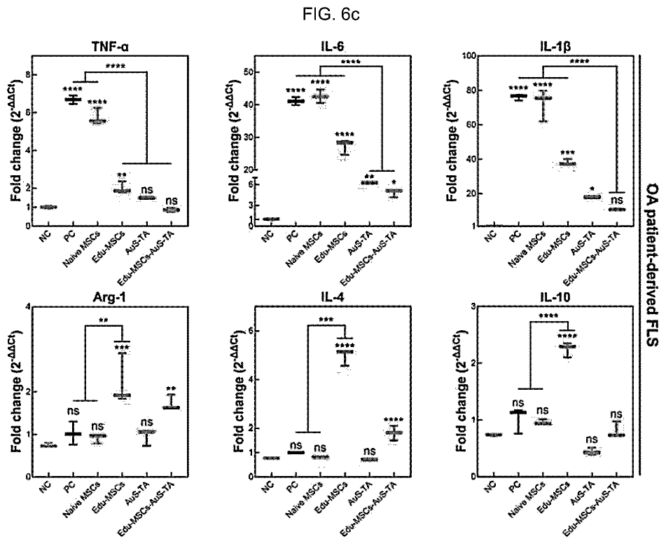

The immunomodulatory ability of Edu-MSCs-AuS-TA was evaluated in FLSs isolated from RA and OA patients (FIG. 6a). FLSs are the major cellular component of the inflamed synovium and the main effector cells in the pathogenesis of arthritis (B. Bartok et al., Immunol. Rev. 9:233-2155, 2010). In arthritis, the number of activated FLSs increases and they become a prominent factor in inflamed joints, thereby upregulating local tissue immunity (N. Bottini et al., Nat. Rev. Rheumatol. 9 (1): 24-33, 2013). Rather than being simply “passive responders” regulated by cytokines and growth factors in the inflammatory environment, FLSs in RA undergo an activated and aggressive phenotype that operates independently of inflammatory stimuli (G. Nygaard et al., Nat. Rev. Rheumatol. 16 (6): 316-333, 2020). Additionally, FLS plays a key role in the development and progression of OA, thereby contributing to joint inflammation and tissue damage through the production of inflammatory cytokines and matrix-degrading enzymes (A. Mobasheri et al., Nat. Rev. Rheumatol. 13:302-311, 2017). Considering the critical role of FLSs in the pathogenesis and phenotypic plasticity of arthritis, the control of FLSs to attenuate arthritis is central to anti-inflammatory treatments for both OA and RA. In the present disclosure, activated FLSs isolated from RA and OA patients were treated with Edu-MSCs-AuS-TA. The upregulated expression of inflammatory cytokines (TNF-α, IL-1β, and IL-6) was downregulated by Edu-MSCs-AuS-TA in both RA patient- and OA patient-derived FLSs, and AuS-TA was the key component in downregulating the expression of inflammatory cytokines (FIGS. 6b and 6c). In RA patient-derived FLS, the use of Edu-MSCs-AuS-TA restored or upregulated the expression of anti-inflammatory cytokines (IL-10, IL-4, and Arg-1) (FIG. 6b). In the case of OA patient-derived FLS, Edu-MSCs-AuS-TA significantly upregulated the expression of Arg-1 and IL-4 (FIG. 6c). The group treated with Edu-MSC alone showed the highest expression of all anti-inflammatory cytokines, whereas naïve MSC showed no effect. These results suggest that Edu-MSCs are key components that upregulate the expression of anti-inflammatory cytokines in OA patient-derived FLS. Therefore, these results indicate that Edu-MSCs-AuS-TA can efficiently increase the immunosuppressive activity of FLS by the synergistic effect of both RA and OA patient-derived FLSs (i.e., a simultaneous increase of anti-inflammatory cytokines by Edu-MSCs and a decrease of proinflammatory cytokines by AuS-TA).

### Example 7: Analysis of Chondroprotective Effect of Edu-MSCs-AuS-TA

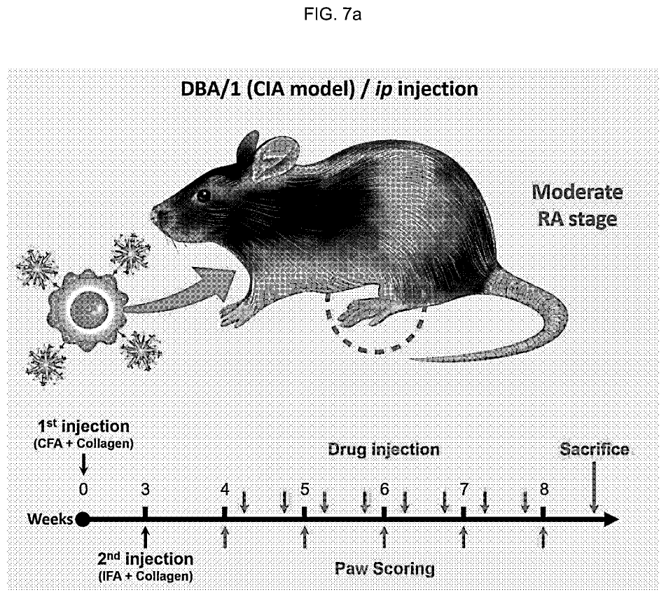

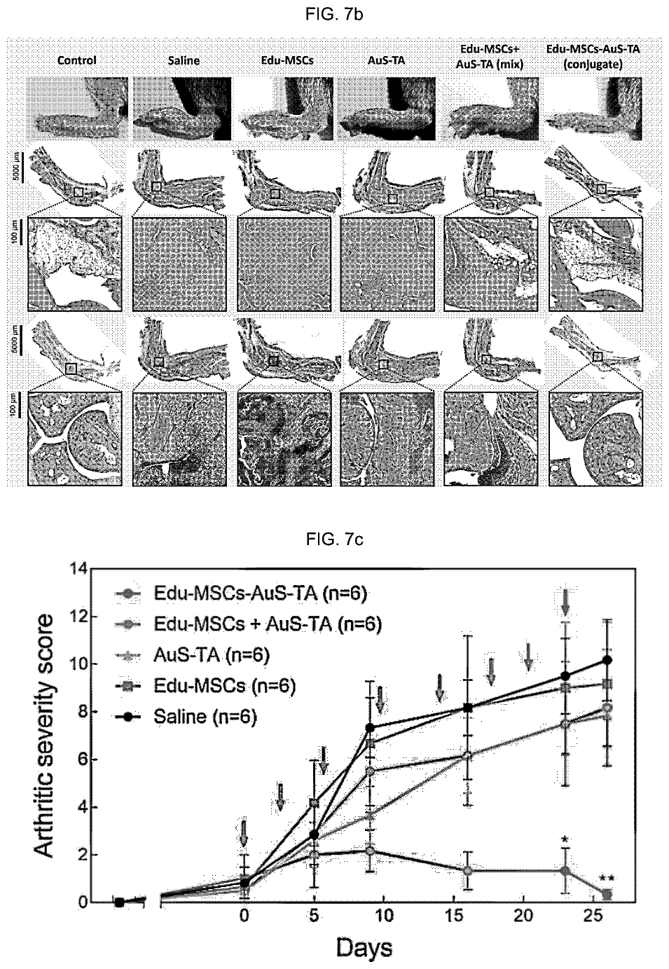

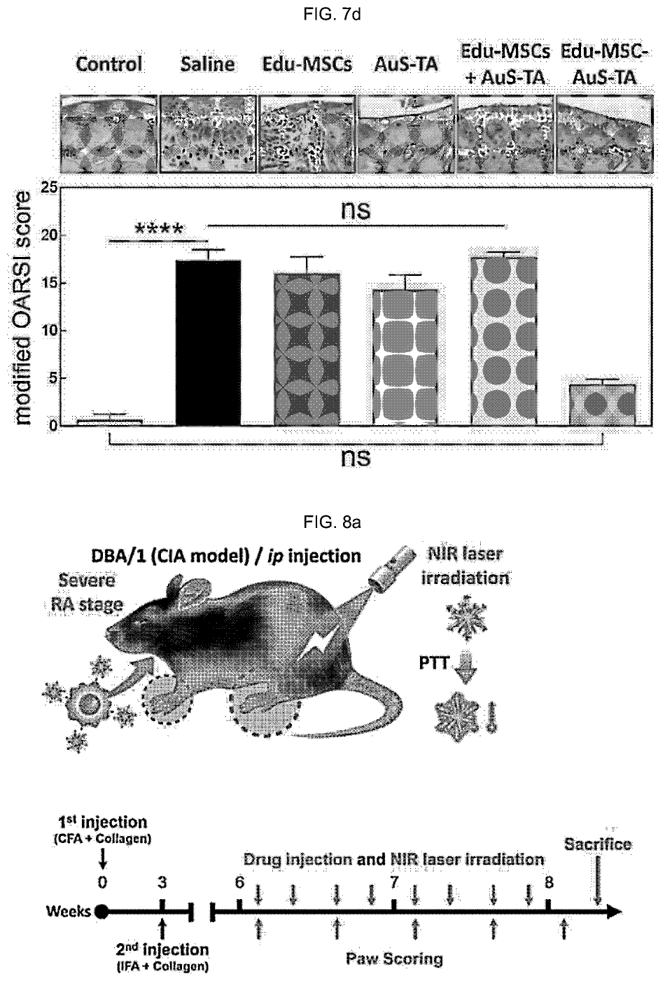

Edu-MSCs-AuS-TA prevents the progression of arthritis and exhibits chondroprotective properties (in vivo). The therapeutic efficacy of Edu-MSCs-AuS-TA in moderate stage of arthritis (arthritis severity score <4) was confirmed in CIA mice (FIG. 7a). The paw edema and paw score of the phosphate-buffered saline (PBS)-treated group (saline group) were significantly increased after 25 days, whereas the paw edema and paw score of the Edu-MSCs-AuS TA (1×10⁶ cells) were significantly decreased (FIGS. 7b and 7c). In contrast, AuS-TA (the same amount of AuS-TA bound to MSCs) and/or Edu-MSCs (1×10⁶ cells) did not sufficiently attenuate the severity of CIA. The CIA severity of the Edu-MSCs-treated CIA model was slightly lower than that of the saline group, but no significant change was observed in the severity score. However, Edu-MSCs-AuS-TA clearly showed an attenuated severity score in the CIA model (FIG. 7c). The therapeutic efficacy of Edu-MSCs-AuS-TA was further evaluated using histological and immunohistochemical analyses (FIGS. 7b and 7d). Hematoxylin and eosin (H&E) staining of mouse ankle joints showed hyperplastic synovium in the inflamed joints in the saline group (FIG. 7b). In contrast, the Edu-MSCs-AuS-TA group had a non-proliferative synovium similar to the normal joint. Furthermore, Edu-MSCs-AuS TA was the only drug that showed chondroprotective properties (as indicated by safranin-O staining) at the same level as the control group (FIGS. 7b and 7d). In other groups, narrowing of joint space and significant depletion of proteoglycans were observed. Additionally, only the Edu-MSCs AuS-TA group showed no significant difference in the modified OARSI scores (the scores based on the structure, cellularity, and chondrocyte replication of cartilage) compared to the control group (FIG. 7d). MSCs have been suggested to play an essential role in tissue repair and tissue regeneration and have anti-inflammatory activity (H. M. Xia et al., Nat. Rev. Mater. 3:174-193, 2018). However, the MSCs in the joints may undergo apoptosis due to harsh conditions, such as low oxygen and nutrient levels including an increased immune response to host antigens, through anoikis (i.e., a form of programmed apoptosis resulting from detachment from the extracellular matrix). Similarly, the group treated with Edu-MSCs alone did not show a sufficient chondroprotective effect, whereas the group treated with AuS-TA showed some efficacy but was still insufficient (FIGS. 7b and 7d). These results indicate that only the step of regulation (either increasing anti-inflammation or decreasing pro-inflammatory responses) alone is not sufficient to induce successful therapeutic efficacy. In contrast, Edu-MSCs-AuS-TA showed significant chondroprotective properties, and it was confirmed that AuS-TA was actively delivered to the inflamed joint by Edu-MSCs-induced Edu-MSCs, thereby maintaining the chondroprotective properties in the inflamed joint. According to the in vivo results above, it is suggested that Edu-MSCs-AuS-TA effectively prevents the progression of RA and cartilage destruction in the CIA model with moderate arthritis (arthritis severity score<4).

### Example 8: Efficacy of Treating Advanced Arthritis Using PTT

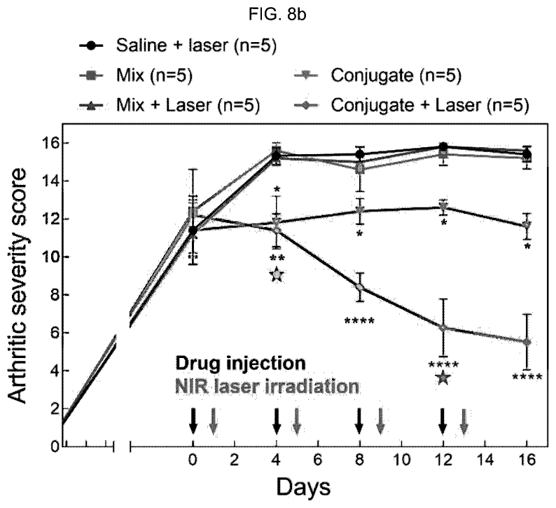

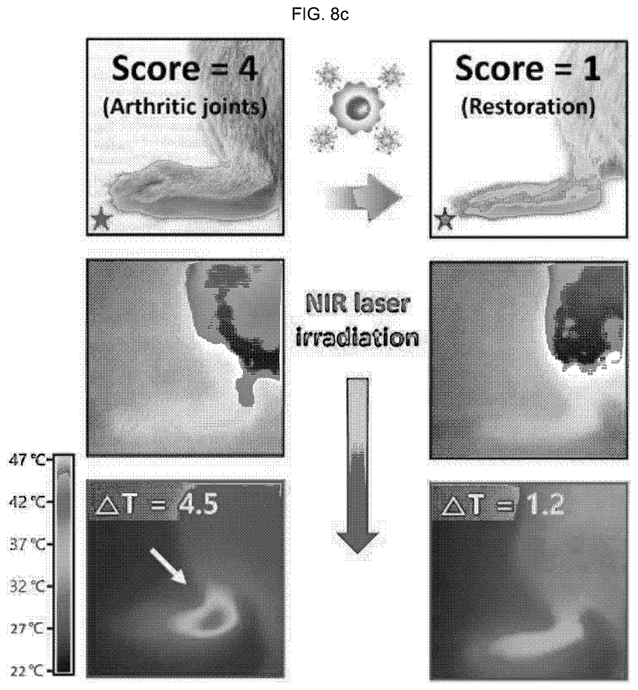

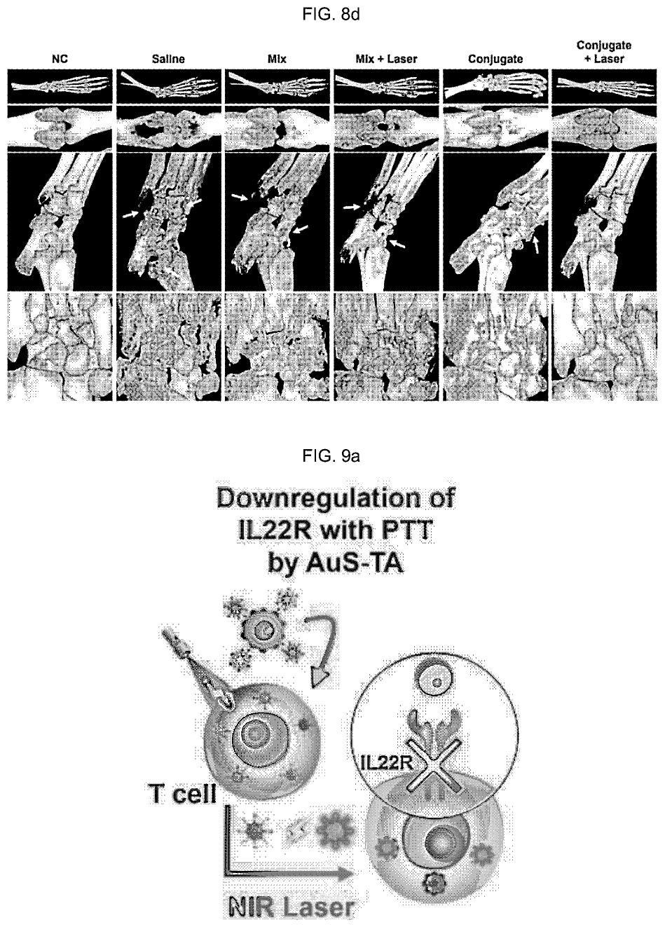

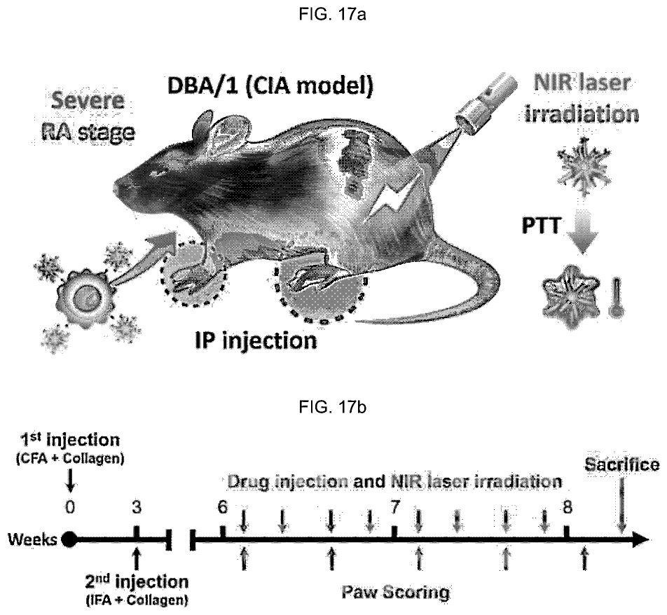

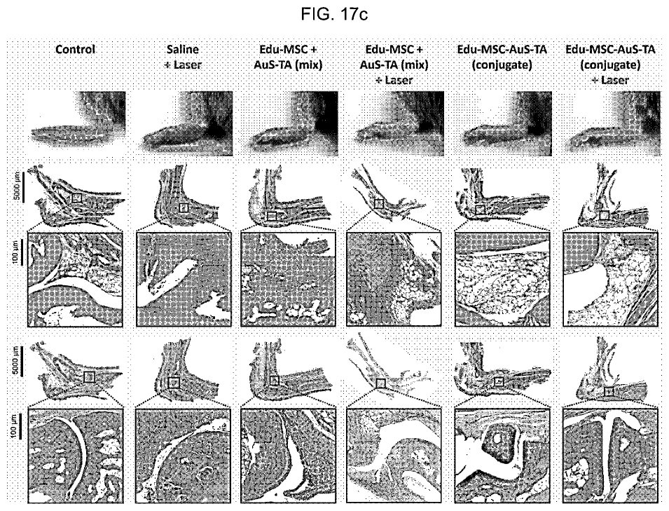

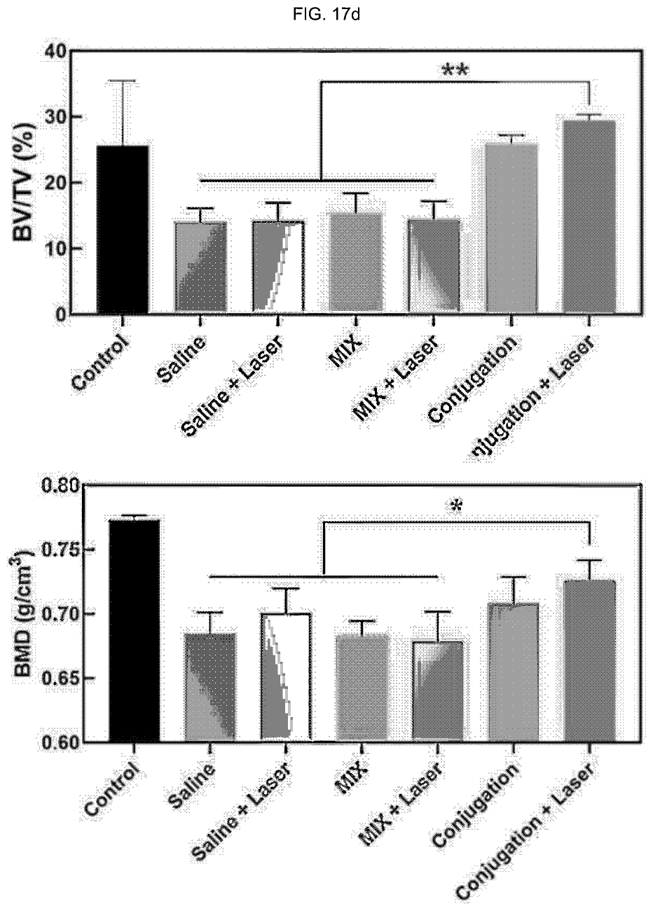

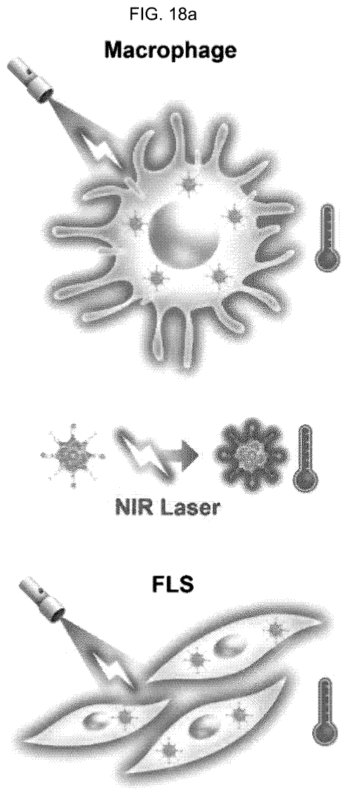

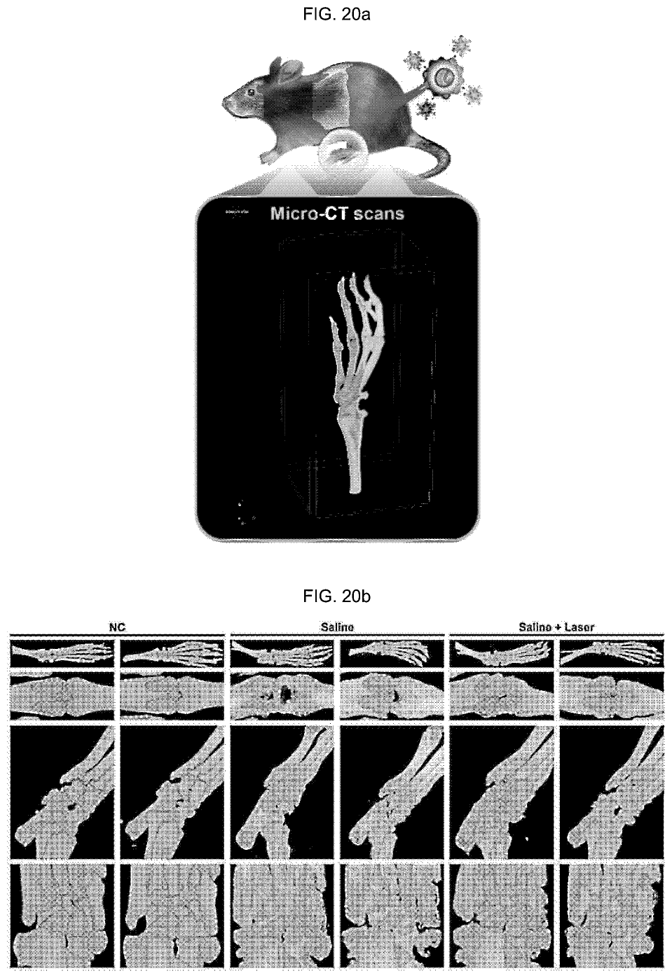

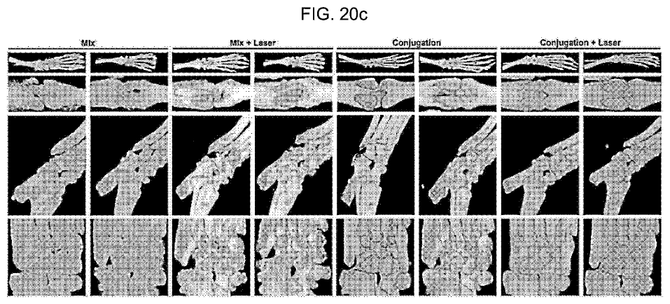

The present inventors treated the CIA model representing the advanced arthritis stage with Edu-MSCs AuS-TA to examine the therapeutic efficacy of Edu-MSCs-AuS-TA in the advanced stage (arthritis severity score >8) (FIG. 8a). As a result, Edu-MSCs-AuS-TA without laser treatment maintained the phenomenon with regard to paw edema and paw score without attenuation in the advanced stage of arthritis (FIG. 8b). This suggests that additional strategies are needed to alleviate advanced arthritis. However, in the group treated with both Edu-MSCs-AuS-TA and NIR laser irradiation, it was shown that paw edema and paw score were significantly reduced (FIG. 8c). The heating of the inflamed joints by laser irradiation was determined according to the severity of the inflamed joints. The joints restored by laser irradiation generated a less amount of heat than before and showed severe inflammation (FIG. 8c). This result is interpreted as showing efficient delivery of AuS-TA by actively targeting MSCs to the joints through enhanced migration ability of Edu-MSCs toward inflammatory mediators upregulated in the inflamed joints at the advanced stage of arthritis. However, after the restoration of joints, the joints were found to have fewer inflammatory mediators, which could override the targeting of AuS-TA, thereby resulting in a decrease in the heating signal. The induction of heating in inflamed joints by laser irradiation provides indirect evidence supporting the accumulation of AuS-TA depending on the severity of arthritis caused by Edu-MSCs in the inflammatory response (FIG. 8c). In addition, three-dimensional micro-computed tomography (micro-CT) images of the hind paw in the CIA model support that Edu MSCs-AuS-TA, in which PTT is included, significantly reduced bone erosion in the joints of CIA mice (FIGS. 8d and 20a). Bone erosion and rough bone surface in CIA mice were effectively restored by treatment with both Edu-MSCs-AuS-TA and laser irradiation. The arthritis severity score of the group treated with Edu-MSCs-AuS-TA showed insufficient efficacy, but the group treated with Edu-MSCs-AuS-TA showed reduced bone erosion and that the bone surface is rough. The calculated bone volume/tissue volume (BV/TV) and bone mineral density (BMD) also showed that the group treated with Edu-MSCs-AuS-TA showed recoveries of bone volume and density to levels similar to those of the negative control group (FIG. 17c). Additionally, the group treated with Edu-MSCs-AuS-TA merely showed hyperplastic synovium (H&E staining) and cartilage regeneration (safranin-O staining) (FIG. 17b). In summary, active delivery of AuS-TA to inflamed joints by Edu-MSCs followed by additional laser irradiation could attenuate the progression of arthritis.

### Example 9: T Cell Regulation Mechanism by PTT

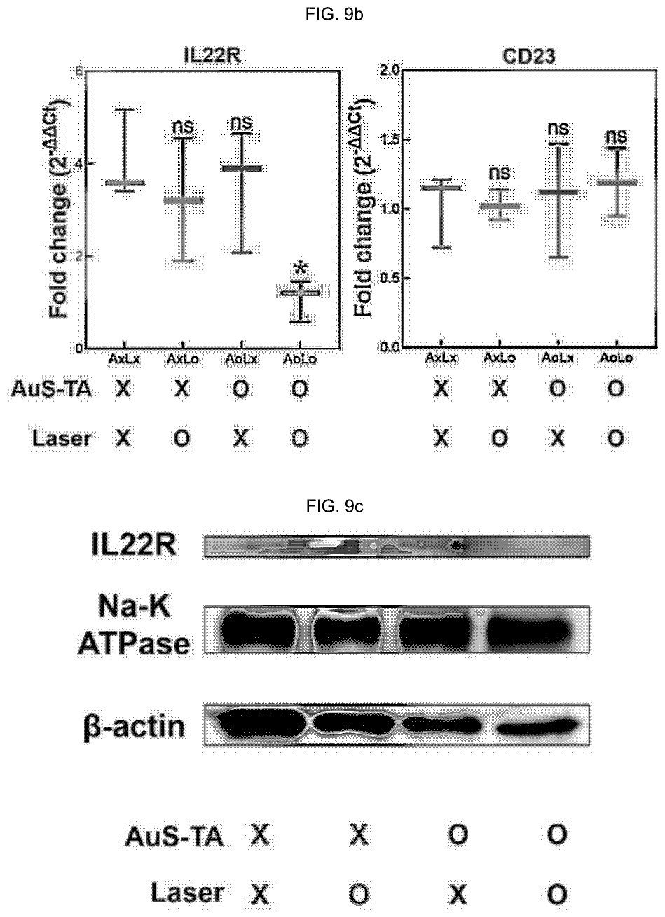

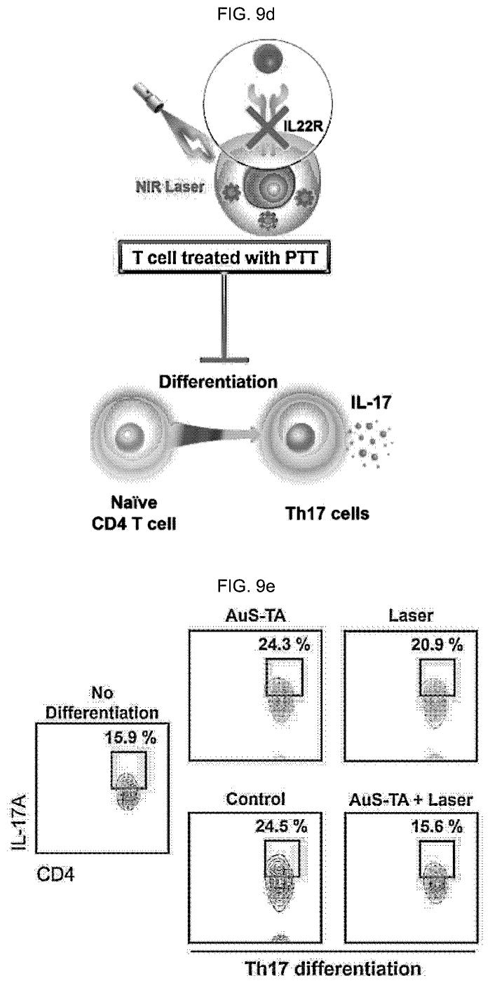

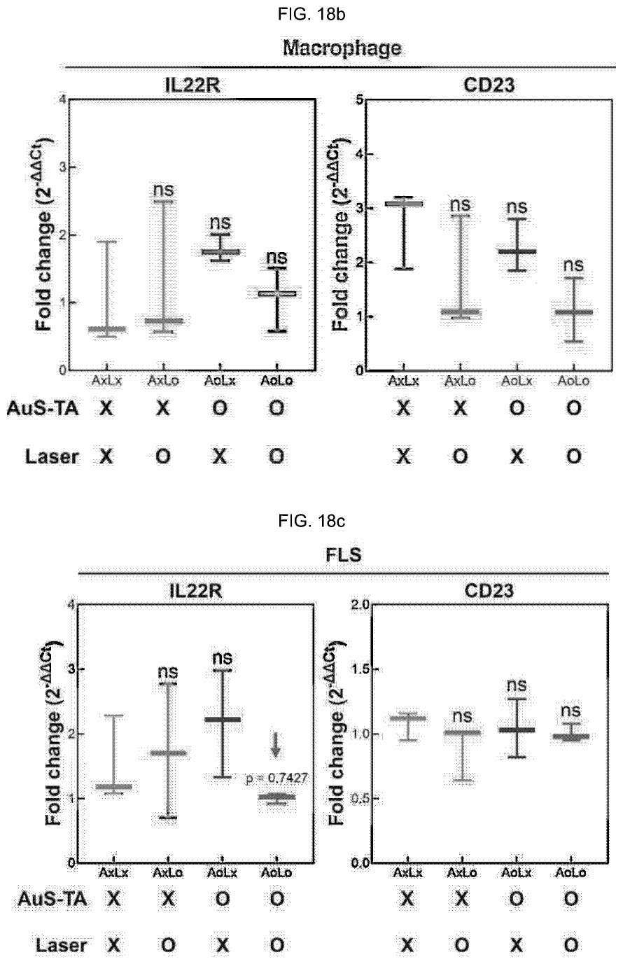

Although the mechanisms of heat-responsive immune regulation have not been clearly elucidated to date, many researchers have focused on studying the mechanisms of apoptosis induced by photothermal therapy, particularly in cancer research (J. R. Melamed et al., ACS Nano 9:6-11, 2015). In the present disclosure, downregulated expression of IL22RA1 in T lymphocytes was first detected via PTT (FIG. 9a). In general, IL22 regulates mucosal barrier defense against microorganisms by upregulating innate immune responses (S. J. Rubino et al., Trends Immunol. 33:112-118, 2012). The present inventors have also confirmed that IL22 is associated with the onset of arthritis. A higher proportion of IL22R expressing CD4⁺ T cells was found in mice with arthritis, whereas no change in population was observed in naïve mice and mice in the early stage of arthritis (H. Lindahl et al., Front. Immunol. 12:618110, 2021). Additionally, neutralization of IL22 in activated CD4⁺ T cells in the CIA model significantly inhibited the expression of IL17, which is a proinflammatory cytokine that primarily contributes to synovitis and joint destruction. However, the role of IL22/IL22R and the potential therapeutic targeting of both proteins in arthritis remain largely unknown and require further investigation. In the present disclosure, T cells isolated from mouse spleen cells were treated with AuS-TA and laser irradiation, and the expression of IL22R in the treated T cells above was significantly reduced at mRNA and protein levels compared to the T cells treated without laser irradiation (FIGS. 9b and 9c). Downregulation of IL22R expression was detected only in the T cells co-treated with AuS-TA and laser, whereas CD23, which is a representative housekeeping gene encoding the receptor, showed no significant difference (FIG. 9b). The inhibition of IL22R by AuS-TA and laser treatment was also confirmed using immunoblotting (FIG. 9c). The inhibition of the IL22/IL22R axis has been shown to inhibit the expression of IL17, which is a cytokine produced primarily by T helper 17 cells (Th17) that play a critical role in the pathogenesis of RA (C. Deligne et al., Osteoarthr. Cartilage 23:1843-1852, 2015). The present inventors hypothesized that downregulation of IL22R expression in T cells by PTT could inhibit Th17 differentiation of naïve CD4 T cells. In order to investigate the above hypothesis, naïve CD4 T cells were treated with AuS-TA and laser irradiation to induce Th17 differentiation (FIG. 9d). The group treated with both AuS-TA and laser showed the smallest Th17 population, similar to the Th17 population of the group where the differentiation was not induced, but the group treated with either AuS-TA or laser alone did not show a significant difference compared to the control group (FIG. 9e). Therefore, the downregulated expression of IL22R using PTT in macrophages and FLS, which play an important role in the pathogenesis of arthritis, was also analyzed (FIG. 18a). Studies on the correlation between the IL22/IL22R axis in FLS or macrophages and the pathogenesis of arthritis also reported that the inhibition of the IL22/IL22R axis in FLS can suppress the production of proinflammatory cytokines in OA- and RA FLS (M. Carrión et al., Rheumatol. (Oxford) 52:2177-2286, 2013). IL22R is widely expressed in macrophages, and the IL-22/IL22R axis is a major activator of a signal transducer and an activator of a transcription (STAT) signaling molecule. Therefore, the IL-22/IL22R axis can induce macrophages into a proinflammatory phenotype through the regulation of STAT1 and STAT5. The expression of IL22R in T cells was significantly downregulated by PTT, but mouse FLS and macrophages (J774) showed no significant difference between the control group and the group with PTT (FIGS. 18b and 18c). Specifically, the FLS treated with PTT showed a slight downregulation of IL22R compared to CD23 expression, and considering the large population of FLS in arthritic joints, the effect of slight downregulation of IL22R expression in FLS by PTT cannot be ignored (FIG. 18c). In the present disclosure, T cells treated with PTT showed specific downregulation of IL22R and subsequent inhibition of Th17 differentiation. Therefore, the PTT-responsive immunomodulation is a key therapeutic strategy for the treatment of advanced arthritis as identified in the present disclosure. In conclusion, the downregulated expression of IL22R in T cells and the inhibition of Th17 differentiation in joint tissues led to the inhibition of IL17 expression and subsequent signaling associated with inflammation and joint destruction.

### Example 10: Innate Immune Response (In Vivo)

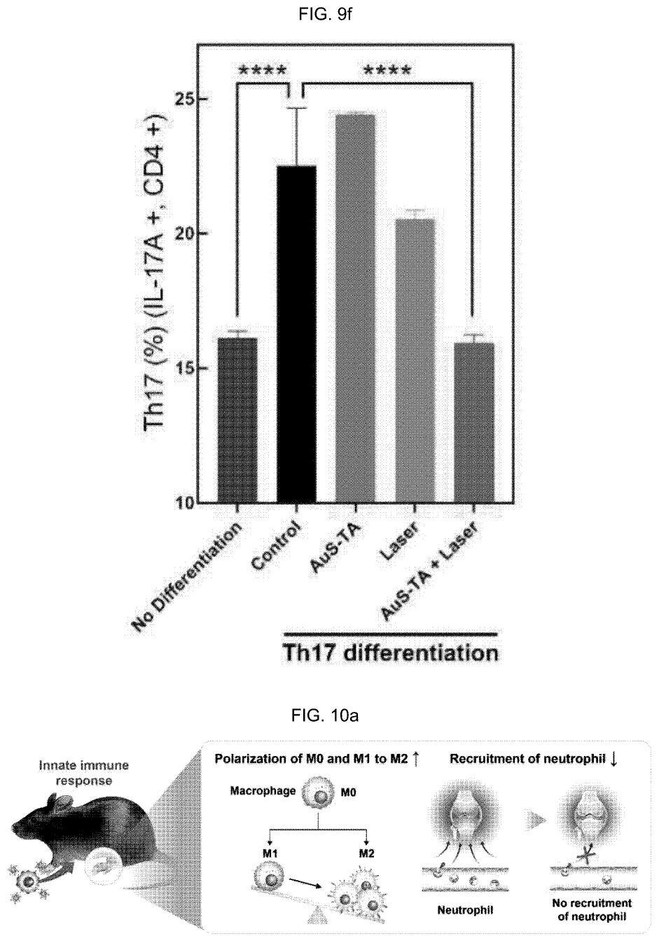

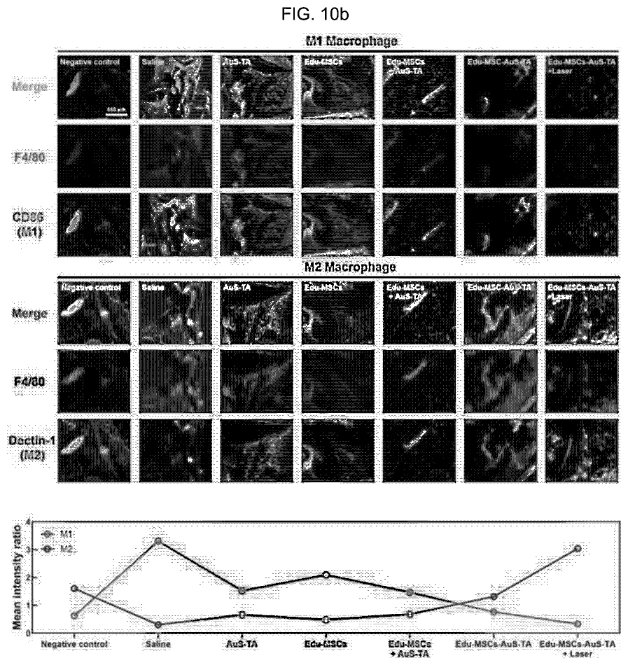

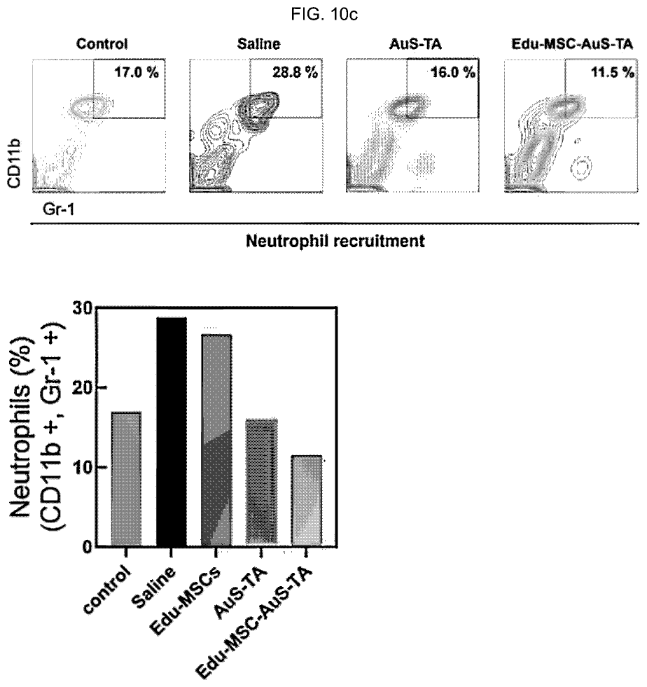

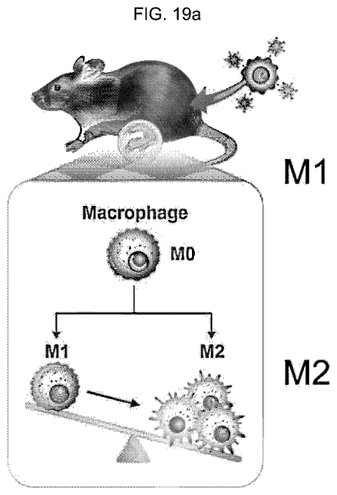

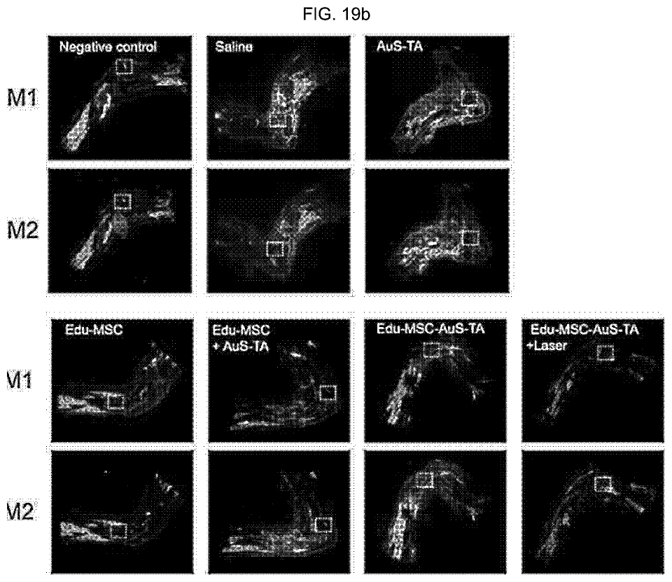

For effective tissue remodeling and repair in arthritis, it is important for macrophages to switch from the proinflammatory M1 phenotype to the antiinflammatory M2 phenotype. The pathogenic characteristic of arthritis is the massive influx of inflammatory cells and macrophages into the joint (FIG. 10a). For example, the number of sublining macrophages within the synovium is an early characteristic of active rheumatoid disease, and the degree of synovial macrophage infiltration correlates with the degree of joint erosion (I. A. Udalova et al., Nat. Rev. Rheumatol. 12:472-485, 2016). Repolarization of macrophages in inflamed joints was investigated using immunofluorescence in histological samples from hind paws (FIGS. 10b, 19a, and 19b). Similar to the therapeutic efficacy of Edu-MSCs-AuS-TA, the repolarization of macrophages from M1 (a reduced level of CD86, which is an M1 marker) phenotype to M2 (an increased level of Dectin-1, which is an M2 marker) phenotype was confirmed to be induced in tissues of all Edu-MSCs AuS-TA-treated groups. Although the group treated with AuS-TA showed a decrease in the level of the M1 phenotype, it was not sufficient to remodel and repair the inflamed joints. Neutrophils, which are the most abundant leukocyte in inflamed joints, are important in the pathogenesis of arthritis, and have the greatest cytotoxic potential due to the release of degradative enzymes and reactive oxygen species. Neutrophils also contribute to cytokine and chemokine cascades that accompany inflammation and regulate immune responses through cell-cell interactions. Moreover, the neutrophils from RA patients have an activated phenotype, and the neutrophils from RA mouse models are important indicators of initiation and progression of diseases (H. L. Wright et al., Nat. Rev. Rheumatol. 10:593-601, 2014). The recruitment of neutrophils isolated from joint tissues of the CIA model of the present disclosure was analyzed by FACS analysis. Edu-MSCs-AuS-TA effectively inhibited neutrophil recruitment into the joints (FIG. 10c). The administered Edu-MSCs-AuS-TA effectively remodeled and repaired inflamed joints through the inhibition of macrophage repolarization and leukocyte recruitment (FIG. 10a). In conclusion, the severity of arthritis was correlated with the M1/M2 macrophage ratio and the number of neutrophils in the synovium, which was similar to the results of previous studies. The M1/M2 macrophage ratio and the number of neutrophils in the synovium were similar to previous studies (J. L. Eyles et al., Blood 112:5193-5201, 2008).

### Example 11: Analgesic Effect of Edu-MSCs-AuS-TA on Arthritis-Associated Pain

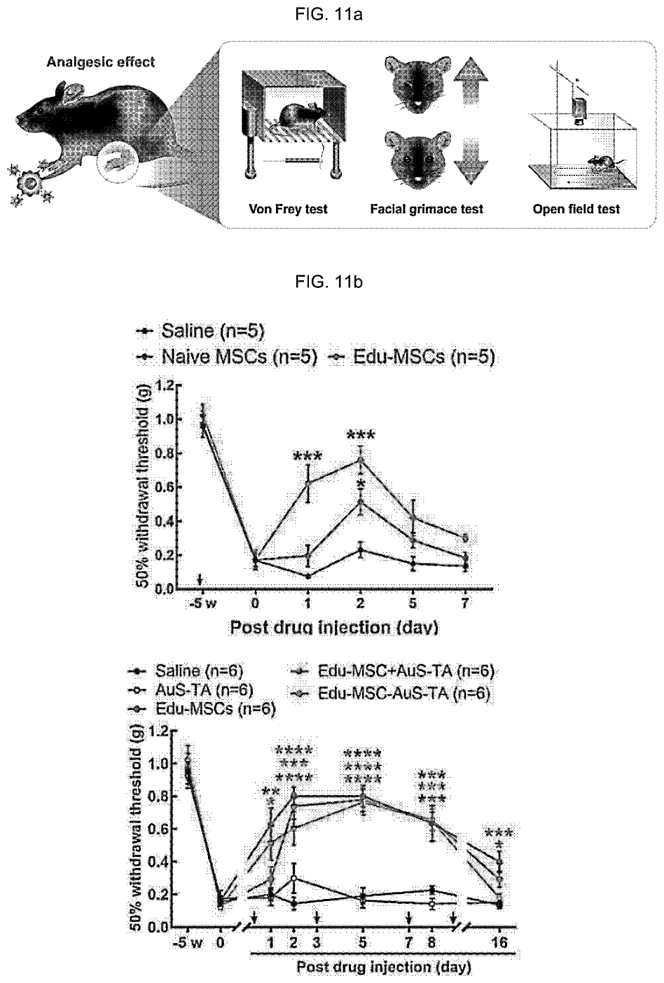

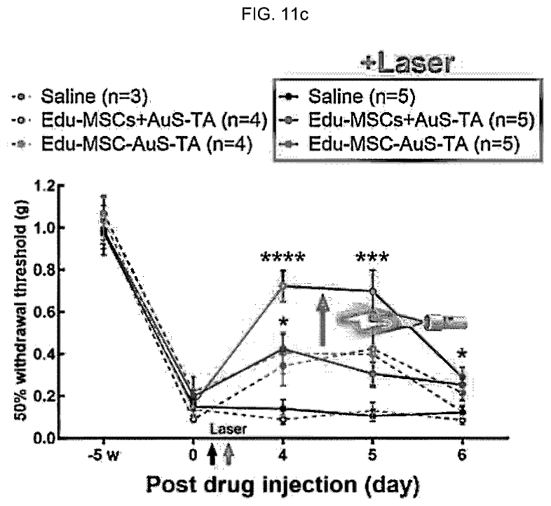

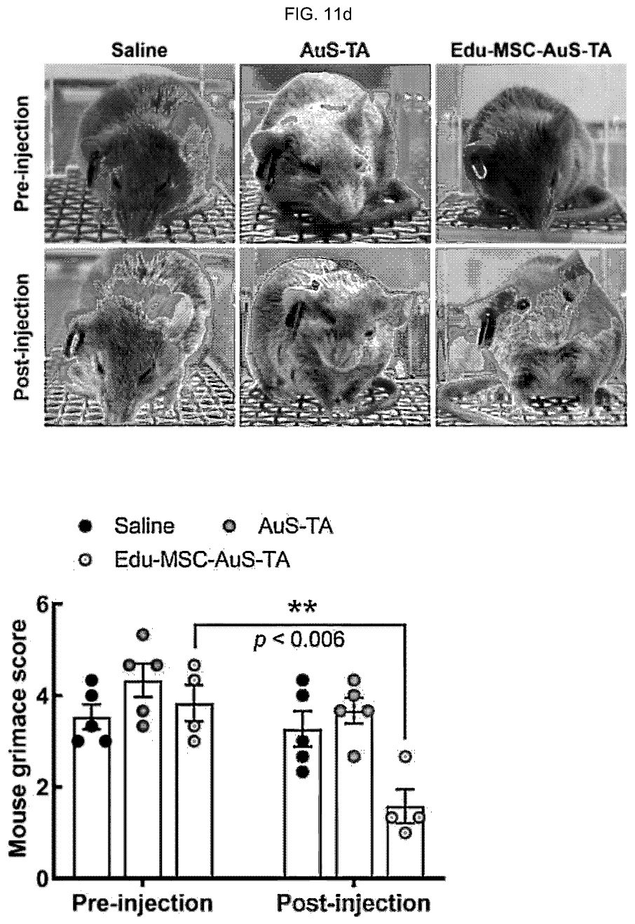

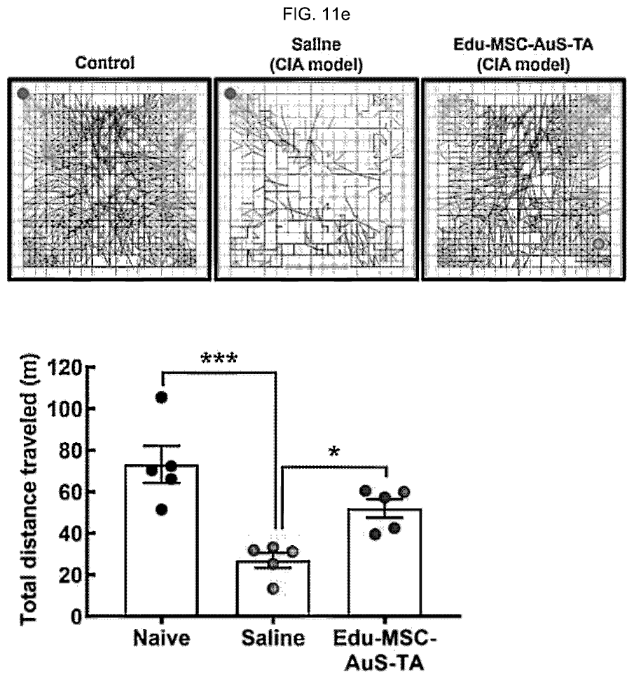

The present inventors examined the analgesic effect of Edu-MSCs-AuS-TA on arthritis-related pain. Pain is the dominant symptom of arthritis and the major reason for patients seeking medical treatment. Arthritis-associated pain may be due to joint inflammation, and the Edu-MSCs-AuS-TA of the present disclosure effectively attenuated joint inflammation in a CIA animal model. In this regard, the analgesic effect of Edu MSCs-AuS-TA was further analyzed using a series of behavioral tests including von Fray, facial grimace, and open field tests (FIG. 11a). As a result, educated MSCs with immunomodulatory functions exhibited enhanced analgesic effects in the CIA model (FIG. 11b). Edu-MSCs-AuS-TA fully restored the threshold value for mechanical stimulation in the CIA model, and multiple administrations of Edu-MSCs-AuS-TA exhibited a long-lasting analgesic effect for about 2 weeks in a fully established RA-associated pain model (FIG. 11b). Compared with the MSC treatment, the Edu-MSCs-AuS-TA treatment alleviated RA-related pain at delayed time points, thus indicating that educated MSCs migrate to the site of inflammation to exhibit an immunomodulatory effect. Importantly, laser irradiation using Edu-MSCs-AuS-TA further enhanced the analgesic effect in the CIA model at advanced stage of arthritis, thus supporting the potent anti-inflammatory effect of TA (FIG. 11c). Additionally, the facial grimacing test and open field test were performed using the CIA model so as to confirm the analgesic effect of Edu-MSCs-AuS-TA on arthritis-related pain. As a result, the administration of Edu-MSCs-AuS-TA significantly improved the facial grimace score and motor activity according to the results of the mechanical allodynia test (FIGS. 11d and 11e). These results suggest that Edu-MSCs-AuS-TA has a potent analgesic effect on arthritis-related pain, thereby enhancing motor activity.

### Example 12: Safety Assay

The present inventors performed a preclinical toxicological evaluation of intraperitoneal injection of MSCs and MSC complexes. Specifically, alanine aminotransferase (ALT), aspartate aminotransferase (AST), creatinine, blood urea nitrogen (BUN), creatinine (Crea), total protein (TP), albumin (Alb), hemoglobin (HGB), and mean corpuscular hemoglobin (MCH) levels were evaluated using blood chemistry tests. For complete blood cell count, hematocrit (HCT), platelets (PLT), white blood cells (WBC), and red blood cells (RBC) were evaluated. As a result, it was found that there was no significant change in any group compared to the control group (FIG. 21a). These results indicate that the injected MSCs and MSC complexes did not cause any notable toxicity in the tested mice. The number of cells injected (1×10⁶ cells) was lower than the number of cells that caused systemic toxicity as observed in previous studies (J. C. Ra et al., Stem Cells Dev. 20:1297-1308, 2011).

The present disclosure has been described with reference to the above-described Examples, but these are merely exemplary, and those skilled in the art will understand that various modifications and equivalent other Examples are possible from this. Therefore, the true technical protection scope of the present disclosure should be determined by the technical idea of the attached patent claims.

The stem cell-derived inflammation targeting complex of the present disclosure, as described above, precisely targets inflammation of the disease and efficiently delivers anti-inflammatory drugs to the site of inflammation; therefore, the complex may be used as a therapeutic agent for inflammatory diseases by promoting cartilage regeneration, enhancing anti-inflammatory effects in both moderate and advanced arthritis, and promoting tissue remodeling. Certainly, the scope of the present disclosure is not limited by these effects.

Although the NIR-responsive stem cell-derived inflammation attenuating complex and use thereof have been described with reference to the specific embodiments, they are not limited thereto. Therefore, it will be readily understood by those skilled in the art that various modifications and changes can be made thereto without departing from the spirit and scope of the present invention defined by the appended claims.

What is claimed is:

1. A stem cell gold nanoparticle complex, wherein gold nanoparticles (AuNPs) loaded with an anti-inflammatory agent are bound to a surface of stem cells.

2. The stem cell gold nanoparticle complex of claim 1, wherein near-infrared (NIR) laser irradiation is used in combination as a photothermal therapy.

3. The stem cell gold nanoparticle complex of claim 1, wherein the near-infrared (NIR) laser irradiation is performed with NIR laser of 800 nm to 900 nm for 3 to 10 minutes.

4. The stem cell gold nanoparticle complex of claim 1, wherein the gold nanoparticles (AuNPs) are coated with PEG on the surface and loaded with steroidal anti-inflammatory drugs by non-covalent bonds.

5. The stem cell gold nanoparticle complex of claim 4, wherein a weight ratio of the steroidal anti-inflammatory drugs to the gold nanoparticles is 2:1 to 5:1.

6. The stem cell gold nanoparticle complex of claim 5, wherein the steroidal anti-inflammatory drug is triamcinolone, hydrocortisone, prednisolone, betamethasone, or dexamethasone.

7. The stem cell gold nanoparticle complex of claim 1, wherein the stem cells are embryonic stem cells, a mesenchymal stem cells, or induced-pluripotent stem cells.

8. The stem cell gold nanoparticle complex of claim 7, wherein the mesenchymal stem cells are bone marrow-derived stem cells, umbilical cord blood-derived stem cells, adipose-derived stem cells, dental pulp-derived stem cells, or peripheral blood-derived stem cells.

9. The stem cell gold nanoparticle complex of claim 1, wherein the stem cells are stem cells that have been educated as lesion-derived cells.

10. The stem cell gold nanoparticle complex of claim 9, wherein the educated stem cells are stem cells that have been cultured by being brought into contact with a culture medium in which the lesion-derived cells are being cultured.

11. A drug delivery carrier comprising the stem cell-gold nanoparticle complex of claim 1 as an active ingredient.

12. A pharmaceutical composition for treating arthritis comprising the stem cell-gold nanoparticle complex of claim 1 as an active ingredient.

13. The pharmaceutical composition of claim 12, wherein the arthritis is rheumatoid arthritis or osteoarthritis.

14. The pharmaceutical composition of claim 12, wherein the arthritis is advanced arthritis with an arthritis severity score of 6 to 8 or higher.

15. The pharmaceutical composition of claim 12, inducing repolarization from M1 macrophages to M2 macrophages.

16. A composition for relieving pain caused by joint inflammation, comprising the stem cell-gold nanoparticle complex of claim 1 as an active ingredient.

17. A method of treating arthritis in a subject in need of comprising administrating therapeutically effective amount of the stem cell-gold nanoparticle complex of claim 1 to the subject.

18. The method of claim 17, wherein the arthritis is rheumatoid arthritis or osteoarthritis.

19. A method of relieving pain caused by joint inflammation in a subject comprising administrating therapeutically effective amount of the stem cell-gold nanoparticle complex of claim 1 to the subject.
